(19) United States
(12) Patent Application Publication
(10) Pub. No.: US 2023/0333119 A1
(43) Pub. Date: Oct. 19, 2023

(54) CIRCULATING TROPHOBLASTS AS A NEW BIOMARKER FOR RISK ASSESSMENT OF MORBIDLY ADHERENT PLACENTATION

(71) Applicant: The Regents of the University of California, Oakland, CA (US)

(72) Inventors: Yazhen Zhu, Los Angeles, CA (US); Hsian-Rong Tseng, Los Angeles, CA (US); Yalda Afshar, Los Angeles, CA (US)

(73) Assignee: The Regents of the University of California, Oakland, CA (US)

(21) Appl. No.: 18/009,663

(22) PCT Filed: Jun. 10, 2021
(86) PCT No.: PCT/US2021/036730

§ 371 Date: Dec. 9, 2022

Related U.S. Application Data
(60) Provisional application No. 63/037,201, filed on Jun. 10, 2020.

(51) Int. Cl. G01N 33/68; G01N 33/569

(52) U.S. Cl. CPC G01N 33/689; G01N 33/56966; G01N 2800/368

(57) ABSTRACT

Methods and kits for isolating at least one of a plurality of single circulating trophoblasts (cTBs) and a plurality of clustered circulating trophoblasts (cTBs) from a blood sample and determining whether a subject has a placenta accrete spectrum (PAS) disorders.

### CROSS-REFERENCE OF RELATED APPLICATION

This application claims priority to U.S. Provisional Application No. 63/037,201 filed Jun. 10, 2020; the entire contents of all of which are hereby incorporated by reference.

### GOVERNMENT SUPPORT

This invention was made with government support under Grant Numbers CA198900, CA235340, and EB026421, awarded by the National Institutes of Health. The government has certain rights in the invention.

### BACKGROUND

### 1. Technical Field

Aspects of the invention relate to methods and kits for isolating at least one of a plurality of single circulating trophoblasts (cTBs) and a plurality of clustered circulating trophoblasts (cTBs) from a blood sample and determining whether a subject has a placenta accrete spectrum (PAS) disorders.

### 2. Discussion of Related Art

Placenta accreta spectrum (PAS) disorders, including placenta accreta, placenta increta, and placenta percreta, are the consequence of abnormal implantation¹, or aberrant invasion and adherence of placental trophoblasts into the uterine myometrium². PAS is associated with significant maternal morbidity because the post-delivery placenta of the fetus does not spontaneously separate and can lead to severe hemorrhage, often leading to an emergency hysterectomy, blood transfusion, and intensive care unit admission^{3,4}. Safe and optimal care of pregnant women with PAS depends on antenatal diagnosis^{3,5}. Current diagnostic modalities for PAS, including serum analytes, ultrasonography, and magnetic resonance imaging (MRI), are effective but not always conclusive, and some options are not readily available in low resource settings⁶⁻⁸. Even in specialized diagnostic units in the United States, around one-third⁹ to half¹⁰ of PAS cases remain undiagnosed during pregnancy. Thus, there is a crucial need to develop novel technologies to improve the antenatal diagnosis of PAS throughout gestation and several blood-based biomarkers—such as plasma protein signatures¹¹, cell-free fetal DNA and cell-free placental mRNA¹²—have been explored for this purpose. Timely detection and diagnosis of PAS provide opportunities to improve prenatal care and minimize maternal and neonatal morbidity by planning delivery in a tertiary-care center with a coordinated team. This has implications both from the individual's risk-stratification and from a broader public health perspective¹³⁻¹⁶. The most important risk factors for PAS are placenta previa (when the placenta implants low and overlays the cervix) and prior cesarean deliveries¹⁷. Given the rising rate of cesarean deliveries, there has been a concomitant 100-fold increase in the incidence of PAS disorders since the 1950s, with a current prevalence of 1 in 500 pregnancies¹⁸. A noninvasive approach for the early detection of PAS is valuable to inform providers and women of their high-risk pregnancy in all health systems, especially in low resource and rural settings without sub-specialists trained in ultrasound^{15,16,19}.

Circulating trophoblasts (cTBs) are placenta-derived trophoblast cells, predominantly of the extra-villous trophoblast (EVT) type, which shed into the maternal circulation during placental implantation and development^{20,21}. Even though rare in numbers, cTBs can be enriched from the maternal circulation. These cells can be used for genetic testing and potentially used as an alternative for non-invasive prenatal testing (NIPT)^{20,22-25}. EVT's function is to migrate and invade at the maternal-fetal interface for normal implantation and placentation; however, when dysfunctional, abnormal invasion occurs, it can lead to PAS^{17,26}. This leads to potential increased cTBs present in the maternal circulation and a means to detect abnormal placental invasion noninvasively. Exploring the utility of cTB enumeration as noninvasive biomarkers for the assessment of excessive EVT invasion may be a promising diagnostic solution to detect PAS throughout gestation.

### INCORPORATION BY REFERENCE

All publications and patent applications identified herein are incorporated by reference in their entirety and to the same extent as if each individual publication or patent application was specifically and individually indicated to be incorporated by reference.

### SUMMARY

An embodiment of the invention relates to a method for determining whether a subject has a placenta accrete spectrum (PAS) disorders, including: isolating at least one of a plurality of single circulating trophoblasts (cTBs) and a plurality of clustered circulating trophoblasts (cTBs) from a blood sample from the subject; detecting at least one of a plurality of isolated single cTBs and a plurality of isolated clustered cTBs from the blood sample; determining at least one of a number of single cTBs from the plurality of isolated single cTBs and a number of clustered cTBs from the plurality of isolated clustered cTBs, wherein a presence of at least one of a predetermined amount of single cTBs and a predetermined amount of clustered cTBs is indicative of an increased probability of the subject having the PAS disorders; and providing a probability of the subject having the PAS disorders based on the determining at least one of a number of single cTBs from the plurality of isolated single cTBs and a number of clustered cTBs from the plurality of isolated clustered cTBs.

An embodiment of the invention relates to a method for determining whether a subject of high risk of placenta accreta spectrum (PAS) disorders has a PAS disorders, including: isolating at least one of a plurality of single circulating trophoblasts (cTBs) and a plurality of clustered circulating trophoblasts (cTBs) from a blood sample from the subject; detecting at least one of a plurality of isolated single cTBs and a plurality of isolated clustered cTBs from the blood sample; determining at least one of a number of single cTBs from the plurality of isolated single cTBs and a number of clustered cTBs from the plurality of isolated clustered cTBs, wherein a presence of at least one of a predetermined amount of single cTBs and a predetermined amount of clustered cTBs is indicative of an increased probability of the subject having the PAS disorders, and wherein a presence of fewer than the predetermined amount of single cTBs and the predetermined amount of clustered cTBs is indicative of the subject having a placenta previa disorders; and providing a probability of the subject having the PAS disorders or the placenta previa disorders based on the determining at least one of a number of single cTBs from the plurality of isolated single cTBs and a number of clustered cTBs from the plurality of isolated clustered cTBs.

An embodiment of the invention relates to a method for capturing at least one of a plurality of single circulating trophoblasts (cTBs) and a plurality of clustered circulating trophoblasts (cTBs) from a blood sample from a subject, including: contacting the blood sample from the subject with a nanostructure-embedded chip such that at least one of the plurality of single circulating trophoblasts (cTBs) and the plurality of clustered circulating trophoblasts (cTBs) from the blood sample are captured by the nanostructure-embedded chip; and detecting the plurality of captured single cTBs and the plurality of captured clustered cTBs from the blood sample.

An embodiment of the invention relates to a kit for capturing at least one of a plurality of single circulating trophoblasts (cTBs) and a plurality of clustered circulating trophoblasts (cTBs) from a blood sample, including: a nanostructure-embedded chip; instructions for capturing at least one of the plurality of single circulating trophoblasts (cTBs) and the plurality of clustered circulating trophoblasts (cTBs) from the blood sample by contacting the blood sample with the nanostructure-embedded chip; and a plurality of reagents for detecting the plurality of captured single cTBs and the plurality of captured clustered cTBs from the blood sample.

### BRIEF DESCRIPTION OF THE DRAWINGS

Further objectives and advantages will become apparent from a consideration of the description, drawings, and examples.

FIGS. 1A and 1B are schematics and micrographs showing use of NanoVelcro Chips for detecting single and clustered circulating trophoblasts (cTBs) in placenta accreta spectrum (PAS) disorder according to an embodiment of the invention.

FIGS. 2A-2F are schematics and micrographs presenting data from the optimization and characterization of NanoVelcro Chips for capturing single and clustered cTBs in clustered cTB blood sample models according to an embodiment of the invention.

FIG. 3 is a schematic depicting a clinical study design according to an embodiment of the invention.

FIGS. 4A and 4B are immunofluorescent images showing the characterization and enumeration of single and clustered cTBs isolated from blood samples collected from pregnant women according to an embodiment of the invention.

FIGS. 5A-5D are data graphs showing results of the comparison of single and clustered cTBs, as well as cTB-clusters in PAS, placenta previa, and normal placentation according to an embodiment of the invention.

FIGS. 6A-6F are data graphs showing receiver operating characteristic (ROC) curves of cTB assay with/without ultrasound according to an embodiment of the invention.

FIGS. 7A-7C are schematics and data graphs of RT-ddPCR assays for detection of trophoblast-specific genes in the cTBs captured by NanoVelcro Chips confirming trophoblast cells of placental origin according to an embodiment of the invention.

FIG. 8 is a schematic showing the entire Nano Velco device according to an embodiment of the invention.

FIG. 9 shows representative images of single cTBs and cTB-clusters of varying cell numbers captured by NanoVelcro Chips according to an embodiment of the invention.

FIGS. 10A-10D are images and data graphs showing the size characterization of cTB-clusters captured by NanoVelcro Chips according to an embodiment of the invention.

FIGS. 11A-11C are data graphs showing counts of single and clustered cTBs as well as cTB-clusters based on stratification of placenta accreta spectrum (PAS) by accreta, increta and percreta according to an embodiment of the invention.

FIGS. 12A-12C show receiver operating characteristic (ROC) curves of single cTBs, clustered cTBs, and cTB-clusters analyzed in different groups according to an embodiment of the invention.

FIGS. 13A-13C are data plots showing the positive predictive values (PPV) and negative predictive values (NPV) as well as sensitivity and specificity for USA cohort, Shenzhen cohort and all cohorts according to an embodiment of the invention.

FIGS. 14A-14F are data graphs showing the counts of single cTBs, clustered cTBs, and cTB-clusters based on gestational age for pregnant women according to an embodiment of the invention.

FIG. 15 is a flowchart for selecting placenta-specific genes and trophoblast-specific genes according to an embodiment of the invention.

FIGS. 16A-16D are representative images of Hematoxylin and Eosin (H&E) staining and immunohistochemistry (IHC) staining of placenta tissues of PAS patients according to an embodiment of the invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

As used throughout, the term “binding agent” refers to any chemical or biologic which facilitates adhesion to a target. In some embodiments according to the invention, the binding agent facilitates capture of single or clustered circulating trophoblasts (cTBs). In some embodiments, the binding agent includes an antibody.

Some embodiments of the invention include a device for capturing a circulating trophoblast. Examples of such devices are described in U.S. Pat. No. 9,140,697, and International Patent Application No. PCT/US2013/043171, each of which is hereby incorporated by reference in its entirety. A further non-limiting example of such a device is a Silicon Nanowire Substrates (SiNWS). In embodiments of the invention, the device includes a substrate; and a plurality of nanowires at least one of attached to or integral with a surface of the substrate such that each nanowire of the plurality of nanowires has an unattached end. In some embodiments, the plurality of nanowires are configured to reversibly attach to self-assembled supramolecular nanoparticles (SMNPs).

In some embodiment, the device for capturing a cell includes a substrate having a nanostructured surface region. Also, in some embodiments, a plurality of binding agents are attached to the nanostructured surface region of the substrate. However, binding agents are not required for the device to bind to target cells. The nanostructured surface region includes a plurality of nanostructures, each having a longitudinal dimension and a lateral dimension. As a sample is placed on the device, biological cells are selectively captured by the binding agents and the plurality of nanostructures acting in cooperation (in embodiments having binding agents). When present, the binding agent or agents employed will depend on the type of biological cell(s) being targeted. Conventional binding agents are suitable for use in some of the embodiments of the present invention. Non-limiting examples of binding agents include antibodies, nucleic acids, oligo- or polypeptides, cellular receptors, ligands, aptamers, biotin, avidin. Coordination complexes, synthetic polymers, and carbohydrates. In some embodiments of the present invention, binding agents are attached to the nanostructured surface region using conventional methods. The method employed will depend on the binding agents and the material used to construct the device. Non-limiting examples of attachment methods include non-specific adsorption to the surface, either of the binding agents or a compound to which the agent is attached or chemical binding, e.g., through self-assembled monolayers or silane chemistry. In some embodiments, the nanostructured surface region is coated with streptavidin and the binding agents are biotinylated, which facilitates attachment to the nanostructured surface region via interactions with the streptavidin molecules.

In some embodiments of the present invention, the nanostructures increase the surface area of the substrate and increase the probability that a given cell will come into contact. In these embodiments, the nanostructures can enhance binding of the target cells by interacting with cellular surface components such as microvilli, lamellipodia, filopodia, and lipid-raft molecular groups. In some embodiments, the nanostructures have a longitudinal dimension that is equal to its lateral dimension, wherein both the lateral dimension and the longitudinal dimension is less than 1 mm, i.e., nanoscale in size. In other embodiments, the nanostructures have a longitudinal dimension that is at least ten times greater than its lateral dimension. In further embodiments, the nanostructures have a longitudinal dimension that is at least twenty times greater, fifty times greater, or 100 times greater than its lateral dimension. In some embodiments, the lateral dimension is less than 1 mm. In other embodiments, the lateral dimension is between 1-500 nm. In further embodiments, the lateral dimension is between 30-400 nm. In still further embodiments, the lateral dimension is between 50-250 nm. In some embodiments, the longitudinal dimension is at least 1 mm long. In other embodiments, the longitudinal dimension is between 1-50 mm long. In other embodiments, the longitudinal dimension is 1-25 mm long. In further embodiments, the longitudinal dimension is 5-10 mm long. In still further embodiments, the longitudinal dimension is at least 6 mm long. The shape of the nanostructure is not critical. In some embodiments of the present invention, the nanostructure is a sphere or a bead. In other embodiments, the nanostructure is a strand, a wire, or a tube. In further embodiments, a plurality of nanostructure contains one or more of nanowires, nanofibers, nanotubes, nano-pillars, nanospheres, or nanoparticles.

An embodiment of the invention relates to a method for determining whether a subject has a placenta accrete spectrum (PAS) disorders, including: isolating at least one of a plurality of single circulating trophoblasts (cTBs) and a plurality of clustered circulating trophoblasts (cTBs) from a blood sample from the subject; detecting at least one of a plurality of isolated single cTBs and a plurality of isolated clustered cTBs from the blood sample; determining at least one of a number of single cTBs from the plurality of isolated single cTBs and a number of clustered cTBs from the plurality of isolated clustered cTBs, wherein a presence of at least one of a predetermined amount of single cTBs and a predetermined amount of clustered cTBs is indicative of an increased probability of the subject having the PAS disorders; and providing a probability of the subject having the PAS disorders based on the determining at least one of a number of single cTBs from the plurality of isolated single cTBs and a number of clustered cTBs from the plurality of isolated clustered cTBs.

An embodiment of the invention relates to the method above, where the predetermined amount of clustered cTBs is 5 clustered cTBs per milliliter of the blood sample, and wherein the predetermined amount of single cTBs is 2 single cTBs per milliliter of the blood sample.

An embodiment of the invention relates to the method above, further including: isolating the plurality of single circulating trophoblasts (cTBs) and the plurality of clustered circulating trophoblasts (cTBs) from the blood sample; detecting the plurality of isolated single cTBs and the plurality of isolated clustered cTBs from the blood sample; determining a number of total cTBs from the plurality of isolated single cTBs and from the plurality of isolated clustered cTBs, wherein a presence of at least a predetermined amount of total cTBs is indicative of an increased probability of the subject having the PAS disorders; and providing a probability of the subject having the PAS disorders based on the determining the number of total cTBs.

An embodiment of the invention relates to the method above, where the predetermined amount is 10 total cTBs per milliliter of the blood sample.

An embodiment of the invention relates to the method above, further including providing a recommended course of action based on the probability of the subject having the PAS disorders.

An embodiment of the invention relates to the method above, where if the probability of the subject having the PAS disorders is increased versus a control, the recommended course of action includes increased surveillance, a transfer and delivery at a tertiary-care facility based, or early uterine-preserving termination of a pregnancy of the subject.

An embodiment of the invention relates to the method above, further including limiting the subject to a subject who is less than 28 weeks of gestational age.

An embodiment of the invention relates to the method above, further including limiting the subject to a subject who is less than 12 weeks of gestational age.

An embodiment of the invention relates to the method above, further including assaying at least one of the plurality of isolated single cTBs and the plurality of isolated clustered cTBs for expression of a biomarker specific to an extra-villous trophoblast.

An embodiment of the invention relates to the method above, where isolating at least one of a plurality of single circulating trophoblasts (cTBs) and a plurality of clustered circulating trophoblasts (cTBs) from the blood sample includes at least one of capturing the plurality of single circulating trophoblasts (cTBs) and capturing the plurality of clustered circulating trophoblasts (cTBs) on a nanostructure-embedded microchip.

An embodiment of the invention relates to a method for determining whether a subject of high risk of placenta accreta spectrum (PAS) disorders has a PAS disorders, including: isolating at least one of a plurality of single circulating trophoblasts (cTBs) and a plurality of clustered circulating trophoblasts (cTBs) from a blood sample from the subject; detecting at least one of a plurality of isolated single cTBs and a plurality of isolated clustered cTBs from the blood sample; determining at least one of a number of single cTBs from the plurality of isolated single cTBs and a number of clustered cTBs from the plurality of isolated clustered cTBs, wherein a presence of at least one of a predetermined amount of single cTBs and a predetermined amount of clustered cTBs is indicative of an increased probability of the subject having the PAS disorders, and wherein a presence of fewer than the predetermined amount of single cTBs and the predetermined amount of clustered cTBs is indicative of the subject having a placenta previa disorders; and providing a probability of the subject having the PAS disorders or the placenta previa disorders based on the determining at least one of a number of single cTBs from the plurality of isolated single cTBs and a number of clustered cTBs from the plurality of isolated clustered cTBs.

An embodiment of the invention relates to the method above, where the predetermined amount of clustered cTBs is 3 clustered cTBs per milliliter of the blood sample, and wherein the predetermined amount of single cTBs is 9 single cTBs per milliliter of the blood sample.

An embodiment of the invention relates to the method above, further including: isolating the plurality of single circulating trophoblasts (cTBs) and the plurality of clustered circulating trophoblasts (cTBs) from the blood sample; detecting the plurality of isolated single cTBs and the plurality of isolated clustered cTBs from the blood sample; determining a number of total cTBs from the plurality of isolated single cTBs and from the plurality of isolated clustered cTBs, wherein a presence of at least a predetermined amount of total cTBs is indicative of an increased probability of the subject having the PAS disorders, and wherein a presence of less than the predetermined amount of total cTBs is indicative of is indicative of the subject having the placenta previa disorders; and providing a probability of the subject having the PAS disorders based on the determining the number of total cTBs.

An embodiment of the invention relates to the method above, where the predetermined amount of total cTBs is 10 total cTBs per milliliter of the blood sample.

An embodiment of the invention relates to the method above, further including providing a recommended course of action based on the probability of the subject having the PAS disorders.

An embodiment of the invention relates to the method above, where if the probability of the subject having the placenta PAS disorders is increased versus a control, the recommended course of action includes increased surveillance, a transfer and deliver to a tertiary-care facility, or uterus-preserving early termination of a pregnancy of the subject.

An embodiment of the invention relates to the method above, further including limiting the subject to a subject who is less than 28 weeks of gestational age (GA).

An embodiment of the invention relates to the method above, further including limiting the subject to a subject who is less than 12 weeks of gestational age (GA).

An embodiment of the invention relates to the method above, further including assaying at least one of the plurality of isolated single cTBs and the plurality of isolated clustered cTBs for expression of a biomarker specific to an extra-villous trophoblast.

An embodiment of the invention relates to the method above, where at least one of the isolating a plurality of single circulating trophoblasts (cTBs) and the isolating a plurality of clustered circulating trophoblasts (cTBs) from the blood sample includes at least one of capturing the plurality of single circulating trophoblasts (cTBs) and capturing the plurality of clustered circulating trophoblasts (cTBs) on a nanostructure-embedded microchip.

An embodiment of the invention relates to a method for capturing at least one of a plurality of single circulating trophoblasts (cTBs) and a plurality of clustered circulating trophoblasts (cTBs) from a blood sample from a subject, including: contacting the blood sample from the subject with a nanostructure-embedded chip such that at least one of the plurality of single circulating trophoblasts (cTBs) and the plurality of clustered circulating trophoblasts (cTBs) from the blood sample are captured by the nanostructure-embedded chip; and detecting the plurality of captured single cTBs and the plurality of captured clustered cTBs from the blood sample.

An embodiment of the invention relates to the method above, further including determining a number of total cTBs from the plurality of captured single cTBs and from the plurality of captured clustered cTBs, wherein a presence of at least a predetermined amount of total cTBs is indicative of an increased probability of the subject having a placenta accrete spectrum (PAS) disorder.

An embodiment of the invention relates to the method above, where the predetermined amount of clustered cTBs is 5 clustered cTBs per milliliter of the blood sample, and wherein the predetermined amount of single cTBs is 2 single cTBs per milliliter of the blood sample.

An embodiment of the invention relates to the method above, further including: capturing the plurality of single circulating trophoblasts (cTBs) and the plurality of clustered circulating trophoblasts (cTBs) from the blood sample; detecting the plurality of captured single cTBs and the plurality of captured clustered cTBs from the blood sample; determining a number of total cTBs from the plurality of isolated single cTBs and from the plurality of isolated clustered cTBs, wherein a presence of at least a predetermined amount of total cTBs is indicative of an increased probability of the subject having the PAS disorders; and providing a probability of the subject having the PAS disorders based on the determining the number of total cTBs.

An embodiment of the invention relates to the method above, where the predetermined amount is 10 total cTBs per milliliter of the blood sample.

An embodiment of the invention relates to the method above, further including providing a recommended course of action based on the probability of the subject having the PAS disorders.

An embodiment of the invention relates to the method above, where if the probability of the subject having the PAS disorders is increased versus a control, the recommended course of action includes increased surveillance, a transfer and delivery at a tertiary-care facility based, or early uterine-preserving termination of a pregnancy of the subject.

An embodiment of the invention relates to the method above, further including assaying at least one of the plurality of isolated single cTBs and the plurality of isolated clustered cTBs for expression of a biomarker specific to an extra-villous trophoblast.

An embodiment of the invention relates to the method above, where the nanostructure-embedded chip includes: a substrate including a plurality of nanowires; and a chaotic mixer overlayed on the substrate.

An embodiment of the invention relates to the method above, where the plurality of nanowires include a plurality of binding agents, the plurality of binding agents specific to an extra-villous trophoblast biomarker.

An embodiment of the invention relates to the method above, where contacting the blood sample with the nanostructure-embedded chip includes passing the blood sample through the nanostructure-embedded chip at a flow rate of between 0.2 ml/h to 1.0 ml/h.

An embodiment of the invention relates to a kit for capturing at least one of a plurality of single circulating trophoblasts (cTBs) and a plurality of clustered circulating trophoblasts (cTBs) from a blood sample, including: a nanostructure-embedded chip; instructions for capturing at least one of the plurality of single circulating trophoblasts (cTBs) and the plurality of clustered circulating trophoblasts (cTBs) from the blood sample by contacting the blood sample with the nanostructure-embedded chip; and a plurality of reagents for detecting the plurality of captured single cTBs and the plurality of captured clustered cTBs from the blood sample.

An embodiment of the invention relates to the kit above, where the instructions for capturing at least one of the plurality of single circulating trophoblasts (cTBs) and the plurality of clustered circulating trophoblasts (cTBs) from the blood sample include passing the blood sample through the nanostructure-embedded chip at a flow rate of between 0.2 ml/h to 1.0 ml/h.

An embodiment of the invention relates to the kit above, where the plurality of reagents for detecting the plurality of captured single cTBs and the plurality of captured clustered cTBs from the blood sample include a reagent for detecting a expression of a biomarker specific to an extra-villous trophoblast.

An embodiment of the invention relates to the kit above, further including instructions for determining a number of total cTBs from the plurality of captured single cTBs and from the plurality of captured clustered cTBs, wherein a presence of at least a predetermined amount of total cTBs is indicative of an increased probability of the subject having a placenta accrete spectrum (PAS) disorder.

An embodiment of the invention relates to the kit above, where further including instructions for providing a recommended course of action based on the probability of the subject having the PAS disorders.

An embodiment of the invention relates to the kit above, where the instructions for providing a recommended course include increased surveillance, a transfer and delivery at a tertiary-care facility based, or early uterine-preserving termination of a pregnancy of the subject.

An embodiment of the invention relates to the kit above, where the nanostructure-embedded chip includes: a substrate including a plurality of nanowires; and a chaotic mixer overlayed on the substrate.

An embodiment of the invention relates to the kit above, where the plurality of nanowires include a plurality of binding agents, the plurality of binding agents specific to an extra-villous trophoblast biomarker.

### Example

“NanoVelcro” Chips were recently developed^{27,28}, and provide immunoaffinity agent-coated nanostructured substrates for improved “stickiness” to such devices, allowing for selective capture of cTBs from pregnant women²⁵, as well as other types of rare cells, e.g., circulating tumor cells (CTCs) from cancer patients²⁹⁻³¹.

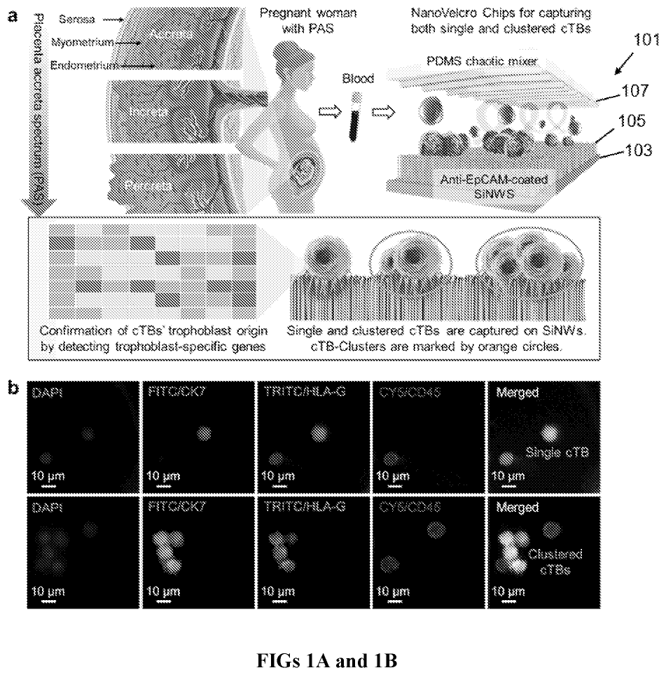

Here, an initial goal was to explore the use of NanoVelcro Chips to detect increased cTBs in maternal circulation as a result of abnormal migration and invasion leading to PAS (FIG. 1a) compared to normal placentation. However, during a pilot study, the presence of aggregates of cTBs in clusters (“cTB-clusters” indicated in the circles in FIG. 1) were discovered, now known as “clustered cTBs” in PAS. To best preserve the intrinsic properties (i.e., morphology and size distribution) of cTB-clusters while retaining the capture performance of the device, a comprehensive optimization investigation was carried out to enable simultaneous detection of both single and clustered cTBs. Using the optimized NanoVelcro Chips, single and clustered cTBs, as well as cTB-clusters in a cohort of 168 pregnant women with clinically confirmed PAS, placenta previa and normal placentation were enumerated. Control studies were performed in 15 healthy non-pregnant female donors. These studies revealed that the counts of single and clustered cTBs, as well as cTB-clusters in PAS are significantly higher than those in non-PAS groups, and the combination of single and clustered cTBs, as well as cTB-clusters can be used to distinguish PAS from normal placentation and/or placenta previa with excellent diagnostic performance in both training, validation, and testing cohorts throughout gestation, particularly early in gestation. In addition, the EVT origin of cTBs captured on NanoVelcro Chips was verified by well-validated immunocytochemistry (ICC) markers and further confirmed by the detection of trophoblast-specific genes, including those of EVT origin, using reverse transcription Droplet Digital polymerase chain reaction (RT-ddPCR). Enumeration of single and clustered cTBs, as well as cTB-clusters can be used for the noninvasive early detection of PAS, holding great promise to improve current diagnostic modalities for PAS detection.

FIGS. 1A and 1B are schematics and micrographs showing use of NanoVelcro Chips for detecting single and clustered circulating trophoblasts (cTBs) in placenta accreta spectrum (PAS) disorder according to an embodiment of the invention. In FIG. 1A, abnormal invasion and adherence of placental trophoblasts into the uterine myometrium, classified into placenta accreta, increta, and percreta based on the severity of the disorder. During implantation and placentation, a small number of cTBs sheds from the placenta into the maternal circulation. NanoVelcro Chip, composed of an overlaid polydimethylsiloxane (PDMS) chaotic mixer and an anti-EpCAM-coated silicon nanowire substrates (SiNWS), was adopted to capture both single and clustered cTBs in maternal blood, allowing for noninvasive detection of PAS disorder. The trophoblast origin of the cTBs was confirmed by detecting trophoblast-specific genes and immunocytochemistry (ICC) staining on the captured cTBs. FIG. 1B shows representative micrographs of ICC staining on a single cTB and clustered cTBs (DAPI+/CK7+/HLA-G+/CD45−) captured by NanoVelcro Chips. Scale bar, 10 μm.

Results

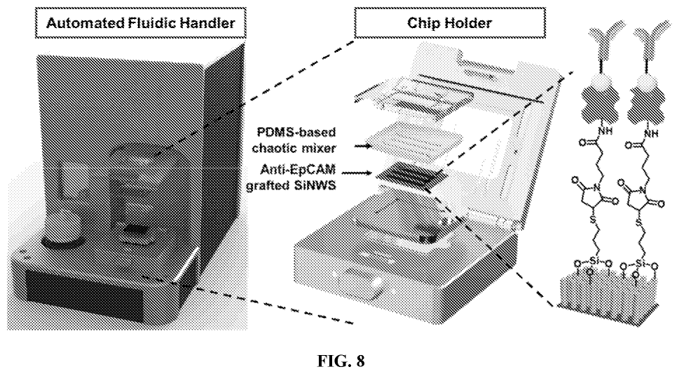

Discovery of clustered cTBs in PAS using NanoVelcro Chips. To test the hypothesis that cTB counts in maternal circulation are elevated in PAS compared to normal placentation, a pilot study to capture cTBs using the NanoVelcro Chip was conducted (FIG. 1a and FIG. 8), which is composed of two functional components, i.e., an anti-EpCAM-grafted silicon nanowire substrate (SiNWS) and an overlaid PDMS chaotic mixer. As seen in FIG. 1A and FIG. 8, the nanochip 101 includes a substrate 103 having a plurality of nanowires 105, and a chaotic mixer 107 overlayed on the substrate 103. FIG. 8 is a schematic showing a NanoVelco device including the nanochip according to an embodiment of the invention. Conceptually, NanoVelcro Assays work analogously like Velcro™: the target cell surfaces covered with nanoscale cell-surface components (a.k.a., microvilli) and the substrate embedded with nanostructures can be regarded as the upper and lower strips of Velcro fastener, respectively. When a cTB contacts the substrate, the microvilli on cTBs' surfaces entangle with the nanostructures on the NanoVelro Chips, introducing increased surface contact areas to facilitate immunoaffinity-mediated cTB capture. Following the previously published procedure for preparation of SiNWS²⁷, Ag nanoparticle-templated wet etching was employed to introduce densely packed silicon nanowires with a high aspect ratio (diameters=100-200 nm, lengths=5-10 μm) onto lithographically patterned silicon wafers. N-hydroxysuccinimide/maleimide chemistry was then adopted to covalently conjugate streptavidin onto the surfaces of SiNWS. Before cTB-capture studies, biotinylated anti-EpCAM were grafted onto SiNWS to confer the specificity to recognize and enrich single and clustered cTBs in blood samples. Here, NanoVelcro Chips pre-coated with anti-EpCAM were utilized to isolate and enumerate cTBs in pregnant women with normal placentation (n=2), placenta previa (n=3), and PAS (n=5). A 4-color ICC protocol²⁵ was developed for the immunofluorescent staining of the captured cTBs. In addition to a conventional cTB marker, i.e., CK7, human leukocyte antigen (HLA)-G—a major histocompatibility tissue-specific antigen that is normally expressed in EVTs^{32,33}—was used to verify the identity of the cTBs and enhance the specificity of this assay. Fluorescence microscopy imaging was adopted to distinguish cTBs (DAPI+/CK7+/HLA-G+/CD45−, FIG. 1b) from background white blood cells (WBCs) (DAPI+/CK7−/HLA-G−/CD45+) immobilized on SiNWS. In pregnant women with PAS, in addition to single cTBs, a phenomenon of clustered cTBs (cTBs present in the cTB-clusters, which were defined as an aggregation of two or more cTBs) was observed. The clustered cTBs shared similar cytomorphology and similar immuno-phenotype with the single cTBs. In this pilot study, no clustered cTBs were detected in pregnant women with normal placentation or placenta previa.

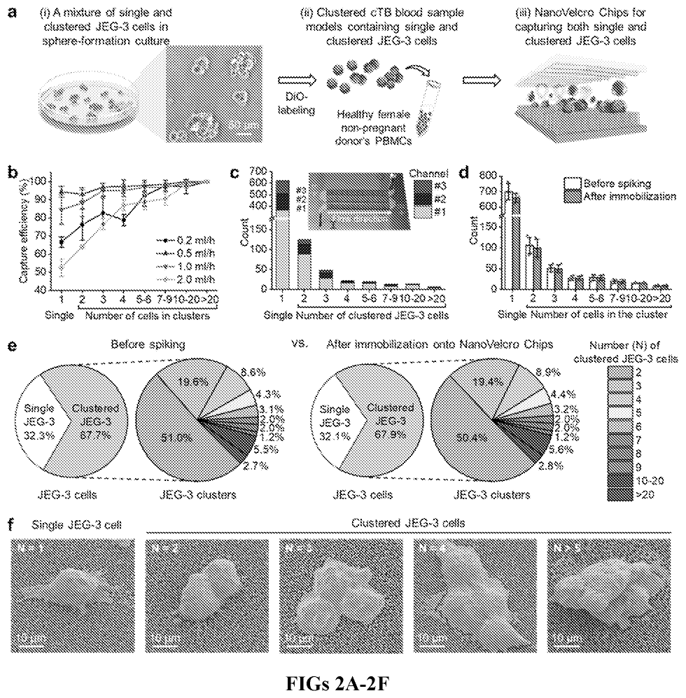

Optimization of NanoVelcro Chips for isolating both single and clustered cTBs. To best preserve the intrinsic properties (including intactness and size distribution) of cTB-clusters while capturing both single and clustered cTBs, the NanoVelcro Chips were optimized according to the general workflow depicted in FIG. 2a. Initially, single and clustered cTBs were obtained by culturing JEG-3 cells (a trophoblast cell line) under sphere-forming conditions³⁴. In a typical sphere-forming JEG-3 cell sample, the majority (67.7%) of cells are present in the form of multidirectional clusters. For the convenience of cell counting by fluorescence microscopy, single and clustered JEG-3 cells in the mixture were labeled with a 3,3′-dioctadecyloxacarbocyanine perchlorate (DiO) green fluorescence dye. Clustered cTB blood sample models were prepared by spiking a healthy non-pregnant female donor's peripheral blood mononuclear cells (PBMCs) (isolated from 2-mL blood) with single and clustered JEG-3 cells. The clustered cTB blood sample models were run through NanoVelcro Chips, followed by the nuclear staining with 4′,6′-diamidino-2-phenylindole (DAPI), then microscopy imaging and enumerating. First, flow rates (0.2, 0.5, 1.0, and 2.0 mL/h) were examined to determine how they affected the efficiencies of capturing single JEG-3 cells and JEG-3 clusters of varying cell numbers. The data summarized in FIG. 2b suggests that 0.5 mL/h is the optimal flow rate with an average capture efficiency of 94% for single JEG-3 and 93-100% for clustered JEG-3 (of cell numbers ranging between 2 and >20). Overall, it was found that (i) NanoVelcro Chips exhibited better capture performance for larger clusters than that observed for single cells and smaller clusters, and (ii) higher flow rates negatively impact the overall capture performance.

FIGS. 2A-2F are schematics and micrographs presenting data from the optimization and characterization of NanoVelcro Chips for capturing single and clustered cTBs in clustered cTB blood sample models according to an embodiment of the invention. FIG. 2A is a general workflow developed for optimization of NanoVelcro Chips for capturing both single and clustered JEG-3 cells. (i) A mixture of single and clustered JEG-3 cells was prepared by culturing under a sphere-formation condition. (ii) Both single and clustered JEG-3 cells were labeled with 3,3′-dioctadecyloxacarbocyanine perchlorate (DiO) and spiked into healthy non-pregnant female donor's peripheral blood mononuclear cells (PBMCs) to prepare clustered cTB blood sample models. (iii) These samples were used for the optimization of NanoVelcro Chips to capture both single and clustered JEG-3 cells. Scale bar, 50 μm. FIG. 2B is a graph showing the performance of capturing single JEG-3 cells and JEG-3 clusters of varying cell numbers at flow rates of 0.2, 0.5, 1.0, and 2.0 mL/h. Data are presented as means±SD of three independent assays. FIG. 2C is a bar graph showing distribution of single JEG-3 cells and JEG-3 clusters along the three microchannels of NanoVelcro Chips at the optimal flow rate of 0.5 mL/h. Data are from one independent experiment. Inset: a photograph of NanoVelcro Chip showing the connected three channels. FIG. 2D is a bar graph showing side-by-side comparison of the distributions of single JEG-3 cells and JEG-3 clusters of varying cell numbers characterized before spiking and after immobilization onto NanoVelcro Chips. Data are presented as means±SD of three independent assays. FIG. 2E are representative pie charts showing the proportions of single and clustered JEG-3 cells, as well as proportions of JEG-3 clusters of varying cell numbers, before spiking (left) and after (right) immobilization. Data are from one independent experiment. FIG. 2F shows SEM images of the single JEG-3 cell (N=1) and clustered JEG-3 cells (N=2, 3, 4, and >5) captured on the SiNWS of NanoVelcro Chips. Scale bar, 10 μm. Data are representatives of three independent assays.

NanoVelcro Chips introducing negligible perturbation to the intrinsic properties of single and clustered cTBs. At the optimal flow rate of 0.5 mL/h, the spatial distribution of single and clustered JEG-3 cells along the three channels on each NanoVelcro Chip was evaluated. As shown in FIG. 2C, 59% (367/622), 22% (139/622), and 19% (116/622) of single JEG-3 cells were captured in the first, second, and third channels, respectively. 71% (175/248), 19% (47/248), and 10% (26/248) of clustered JEG-3 were captured in the first, second, and third channels, respectively. The predominant distribution in the first two channels (81-90%) suggests that NanoVelcro Chips have sufficient channel length to capture both single and clustered JEG-3 cells. Finally, how the capture process in NanoVelcro Chips could perturb the intrinsic properties (i.e., intactness and size distribution) of JEG-3 clusters was investigated. FIG. 2D shows a side-by-side comparison of the distribution of single JEG-3 cells and JEG-3 clusters of varying cell numbers before spiking and after immobilization onto NanoVelcro Chips. This is also depicted in the representative pie charts in FIG. 2E. Therefore, the distribution observed after immobilization is similar to that before spiking, suggesting that NanoVelcro Chips introduces negligible perturbations to the intrinsic properties of cTB-clusters. To better elucidate how the Velcro-like operating mechanism^{27,30} facilitates immunoaffinity-mediated capture of single and clustered JEG-3 cells onto SiNWS, scanning electron microscopy (SEM) imaging was employed to characterize the interfaces between single and clustered JEG-3 cells and SiNWS after capture. FIG. 2F shows representative SEM images of a single JEG-3 cell (N=1) and clustered JEG-3 cells (N=2, 3, 4, and >5) captured on SiNWS of NanoVelcro Chips. Entangled interactions between long microvilli of JEG-3 cells and densely packed silicon nanowires on SiNWS, i.e., the characteristic “Velcro-like” interactions at the interfaces between single and clustered JEG-3 cells and SiNWS were seen. In conclusion, NanoVelcro Chips introduced negligible perturbations to the morphology of single and clustered JEG-3 cells, suggesting that the Velcro-like operating mechanism, originally developed to facilitate immunoaffinity-mediated capture of single cTBs, can also be effectively adopted for immunoaffinity-mediated capture of clustered cTBs.

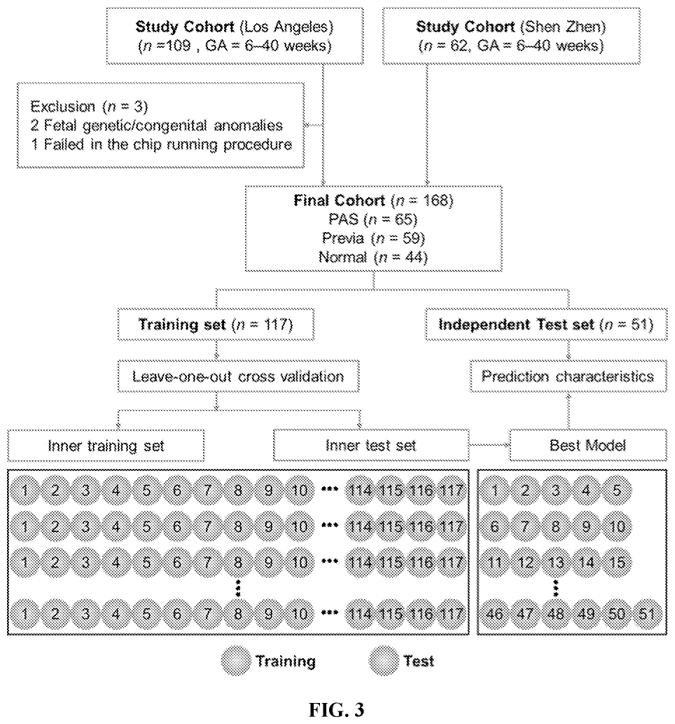

Characterization of single and clustered cTBs isolated from blood samples of pregnant women. Using the above optimized experimental conditions, NanoVelcro Chips were employed to detect and enumerate single and clustered cTBs, as well as cTB-clusters in clinical samples following the streamlined workflow (see Methods). FIG. 3 is a schematic depicting a clinical study design according to an embodiment of the invention. Among the 171 eligible pregnant women recruited in this study, three subjects were excluded due to fetal genetic/congenital anomalies, or technical failure. Blood samples from 168 individuals in four cohorts were collected, (i) PAS cohort: prenatally suspected and subsequently pathologically confirmed PAS patients (n=65, mean age=36 years old (yo)); (ii) placenta previa cohort: clinically diagnosed placenta previa patients (n=59, mean age=35 yo); (iii) normal placentation cohort: pregnant women with clinically confirmed normal placentation (n=44, mean age=37 yo); and (iv) healthy non-pregnant female donors (n=15, mean age=29 yo). The demographic information of these cohorts is provided in Table 1 and Table 2. Characterization and enumeration were performed by an investigator blinded to all clinical information. For each blood sample, PBMCs were obtained from 2 mL of whole blood (via gradient centrifugal depletion of red blood cells) and then processed through NanoVelcro Chips. After performing the 4-color ICC²⁵ to stain DAPI, CK7, HLA-G, and CD45, single and clustered cTBs (DAPI+/CK7+/HLA-G+/CD45−) were identified from background WBCs (DAPI+/CK7−/HLA-G−/CD45+) under a fluorescence microscope.

TABLE 1
Clinical information for pregnant women (n = 168) enrolled in the study.

| | | Non-PAS (n = 103) | | p value |
|---|---|---|---|---|
| | | Placenta | Normal | (PAS |
| | PAS | previa | placentation | versus |
| Characteristics | (n = 65) | (n = 59) | (n = 44) | non-PAS) |
| Median maternal | 36 (23-44) | 35 (19-54) | 37 (22-45) | 0.877 |
| age (range)-yo | | | | |
| Pre-pregnancy | 24 (16-49) | 23 (17-32) | 24 (19-44) | 0.486 |
| BMI* (range) | | | | |
| In vitro fertilization | | | | |
| (IVF)-n (%) | | | | 0.583 |
| Yes | 14 (21.5) | 11 (18.6) | 15 (34.1) | |
| No | 51 (78.5) | 48 (81.4) | 29 (65.9) | |
| Gravidity-n (%) | | | | 0.018 |
| 1 | 8 (12.3) | 7 (11.9) | 15 (34.1) | |
| 2 | 11 (16.9) | 16 (27.1) | 15 (34.1) | |
| ≥3 | 46 (70.8) | 36 (61.0) | 14 (31.8) | |
| Parity-n (%) | | | | 0.029 |
| 0 | 10 (15.4) | 13 (22.0) | 20 (45.5) | |
| 1 | 26 (40.0) | 19 (32.2) | 21 (47.7) | |
| ≥2 | 29 (44.6) | 27 (45.8) | 3 (6.8) | |
| Previous cesarean | | | | |
| delivery (CD)-n (%) | | | | 0.001 |
| 0 | 15 (23.1) | 21 (35.6) | 32 (72.7) | |
| 1 | 30 (46.1) | 20 (33.9) | 11 (25.0) | |
| ≥2 | 20 (30.8) | 18 (30.5) | 1 (2.3) | |

*BMI = body mass index (kg/m²)

TABLE 2
Clinical information for healthy non-pregnant female donors.

| Sample ID | Maternal age | Gravidity | Parity | Site |
|---|---|---|---|---|
| HFD01 | 40 | 1 | 1 | Los Angeles |
| HFD02 | 39 | 0 | 0 | Los Angeles |
| HFD03 | 42 | 4 | 2 | Los Angeles |
| HFD04 | 41 | 1 | 1 | Los Angeles |
| HFD05 | 30 | 0 | 0 | Los Angeles |
| HFD06 | 38 | 1 | 1 | Los Angeles |
| HFD07 | 40 | 2 | 2 | Los Angeles |
| HFD08 | 42 | 2 | 2 | Los Angeles |
| HFD09 | 28 | 0 | 0 | Los Angeles |
| HFD10 | 26 | 0 | 0 | Los Angeles |
| HFD11 | 28 | 0 | 0 | Los Angeles |
| HFD12 | 27 | 0 | 0 | Los Angeles |
| HFD13 | 39 | 3 | 1 | Los Angeles |
| HFD14 | 33 | 0 | 0 | Los Angeles |
| HFD15 | 28 | 0 | 0 | Los Angeles |

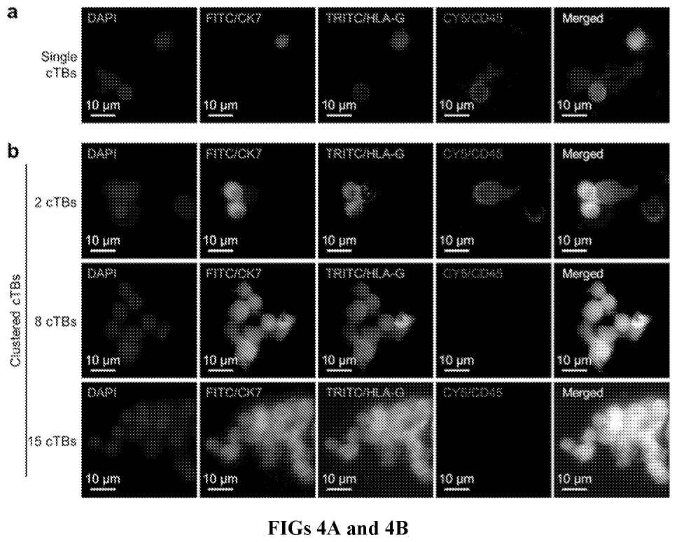

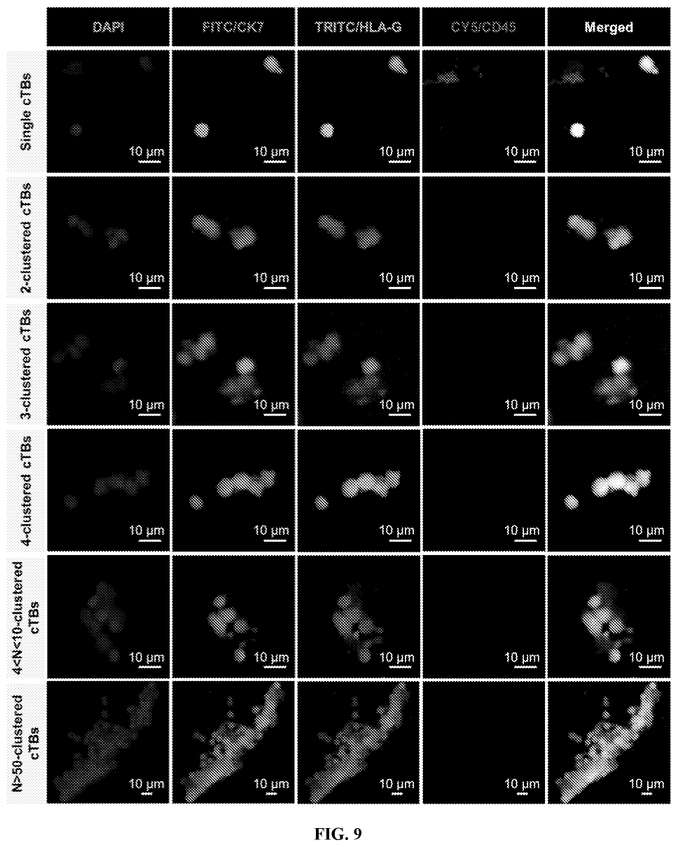

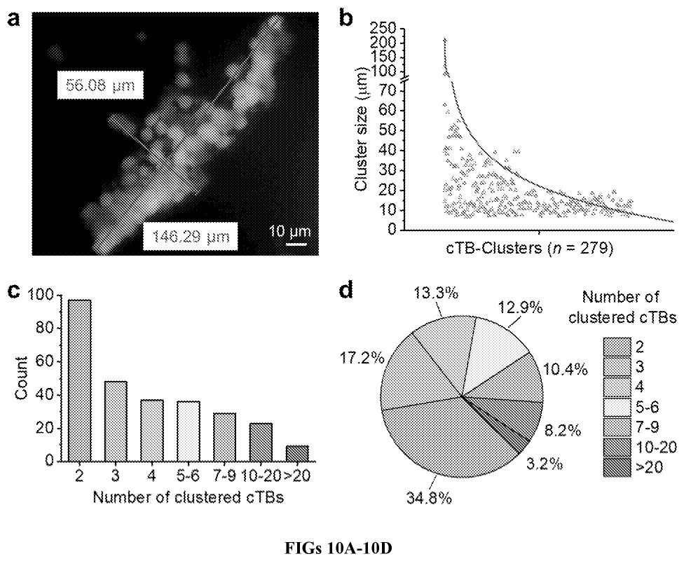

Representative micrographs of a single cTB, as well as clustered cTBs in different sizes of cTB-clusters including of 2, 8, and 15 cells isolated from blood samples of pregnant women with PAS, are shown in FIGS. 4A and 4B. Additional images of single cTBs as well as clustered cTBs in different sizes of cTB-clusters are provided in FIG. 9. The morphology of single cTBs is usually round and smooth with diffuse nuclear DAPI staining and uniform cytoplasmic CK7 staining. cTB-clusters are characterized as an aggregation of two or more cTBs, and the clustered cTBs are the cTBs present in a cTB-cluster. Both the single and clustered cTBs exhibit cytoplasmic and membrane HLA-G staining. The overall size of cTB-clusters (n=279) ranges from 7 μm to 210 μm (as seen in FIGS. 10A and 10B) depending on the configuration and numbers of cTBs in each cluster. The distribution and proportion of cTB-clusters of varying numbers of cells are summarized in FIGS. 10C and 10D. Despite their size differences, cTB-clusters are composed of small round cTBs with a high level of homogeneity.

FIGS. 4A and 4B are immunofluorescent images showing the characterization and enumeration of single and clustered cTBs isolated from blood samples collected from pregnant women according to an embodiment of the invention. Representative immunofluorescent images of single (FIG. 4A) and clustered cTBs (FIG. 4B) in different sizes of cTB-clusters shown at 40× magnification. Scale bar, 10 μm.

FIG. 9 shows representative images of single cTBs and cTB-clusters of varying cell numbers captured by NanoVelcro Chips according to an embodiment of the invention. Scale bar, 10 μm.

FIGS. 10A-10D are images and data graphs showing the size characterization of cTB-clusters captured by NanoVelcro Chips according to an embodiment of the invention. FIG. 10A shows the size of a cTB-cluster was measured along the longest axis and width perpendicular to that axis across the cTB-clusters, defined as:

\sqrt{(longest axis)×(perpendicular width)}.

Scale bar, 10 μm. FIG. 10B is a plot showing the cluster size range of cTB-clusters (n=279). FIGS. 10C and 10D are bar and pie charts, respectively, showing the distribution and proportion of cTB-clusters (n=279) with varying numbers of cells, respectively.

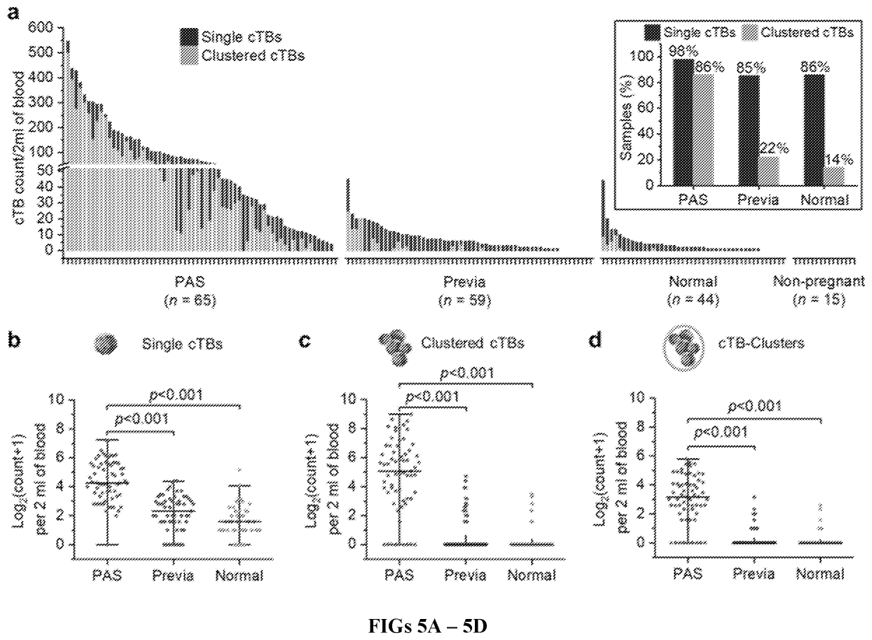

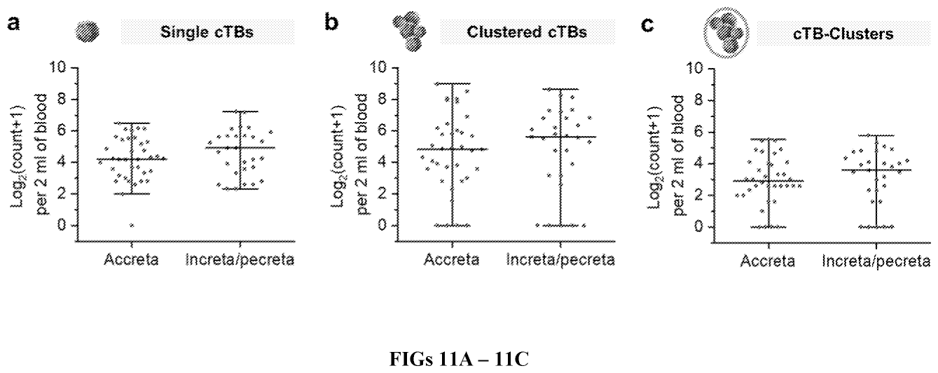

Single and clustered cTBs as a putative biomarker for detecting PAS from placenta previa and normal placentation. Blood samples from a final cohort of 168 pregnant women were subjected to NanoVelcro Chip assays and analyzed according to the combination of single and clustered cTBs, as well as cTB-clusters. The final cohort included 65 PAS, 59 placenta previa, and 44 normal placentation. The enumeration results (cTB count per 2 mL of blood) of single cTBs (blue bar) and clustered cTBs (orange bar) from each blood sample, are summarized in FIG. 5A. Overall, single cTBs are detected in the majority of pregnant women, with a detection rate of 98%, 85%, and 86% in the groups of PAS, placenta previa, and normal placentation, respectively (FIG. 5A inset). Notably, the detection rates of clustered cTBs (i.e., cTBs present in cTB-clusters) in the PAS group (86%) was statistically significantly higher (Chi-square test, p<0.001) than the placenta previa group (22%) and the normal placentation group (14%). Of the 6 PAS samples without clustered cTBs, 3 samples were from women with a focal accreta confirmed intraoperatively, a less severe phenotype of PAS. This result raises the question of the correlation between cTB enumeration and severity of the disease. Therefore, the counts of single and clustered cTBs were first compared, as well as cTB-clusters among the subtypes of PAS (i.e., accreta versus increta and percreta). Results summarized in FIGS. 11A-11C indicated that despite a trend showing increased numbers of cTBs or cTB-clusters with more severe disease, the comparison is not statistically significant (p>0.05) in the current study. cTBs were not detected in healthy non-pregnant female donors. The counts of single and clustered cTBs, as well as cTB-clusters among the three study groups were compared (i.e., PAS, Previa, and Normal) in FIGS. 5B-5D. Significantly higher counts of single and clustered cTBs, as well as cTB-clusters were observed in the PAS group compared to those found in the Previa and Normal groups, suggesting the potential role of cTBs and cTB-clusters in distinguishing PAS from placenta previa, and normal placentation.

FIGS. 5A-5D are data graphs showing results of the comparison of single and clustered cTBs, as well as cTB-clusters in PAS, placenta previa, and normal placentation according to an embodiment of the invention. FIG. 5A shows counts of single and clustered cTBs per 2 mL of blood for all participants enrolled in the study (n=183). Pregnant women (n=168) are divided into three groups (i.e., PAS, placenta previa, and normal placentation) based on clinical diagnosis and sorted within groups based on total cTB count (single cTB count plus clustered cTB count). Non-pregnant indicates the control cohort of healthy non-pregnant female donors (n=15). The inset shows the percentages of pregnant women with detectable single and clustered cTBs in PAS, placenta previa, and normal placentation. Counts of: single cTBs (FIG. 5B), clustered cTBs (FIG. 5C), and cTB-clusters (FIG. 5D) per 2 mL of blood for pregnant women with PAS (n=65), placenta previa (n=59), and normal placentation (n=44) are shown. Counts were log 2-transformed. The two shorter horizontal lines denote the 25-75% interquartile ranges (IQR) and the longer horizontal lines in between denote the median. Data are expressed as Mean±SE for single cTBs (FIG. 5B): PAS (4.4±0.2), placenta previa (2.2±0.2), normal placentation (1.6±0.2); clustered cTBs (FIG. 5C): PAS (4.7±0.3), placenta previa (0.6±0.2), normal placentation (0.3±0.1); and cTB-clusters (FIG. 5D): PAS (3.0±0.2), placenta previa (0.3±0.1), normal placentation (0.2±0.1).

FIGS. 11A-11C are data graphs showing counts of single and clustered cTBs as well as cTB-clusters based on stratification of placenta accreta spectrum (PAS) by accreta, increta and percreta according to an embodiment of the invention. FIG. 11A shows single cTBs, FIG. 11B shows clustered cTBs, and FIG. 11C shows cTB-clusters. Counts were log 2-transformed. The two shorter horizontal lines denote the 25-75% interquartile ranges (IQR) and the longer horizontal lines in between denote the median. Data are expressed as Mean±SE for single cTBs (FIG. 11A): accreta (4.2±0.2), increta and pecreta (4.6±0.3); clustered cTBs (FIG. 11B): accreta (4.5±0.4), increta and pecreta (4.9±0.5); and cTB-clusters (FIG. 11C): accreta (2.9±0.3), increta and pecreta (3.1±0.3).

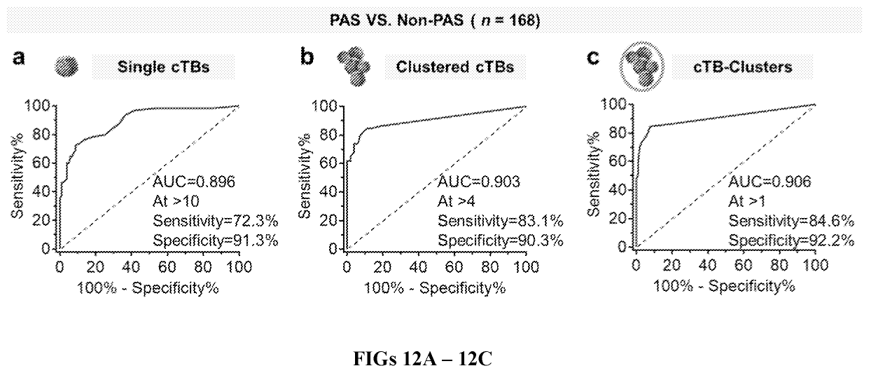

To integrate the enumeration results of single and clustered cTBs, as well as cTB-clusters into a statistically robust prediction model, each variable was screened to determine if it served as a statistically significant univariate predictor of PAS status. Receiver operating characteristic (ROC) analysis was conducted to assess the diagnostic performance of the single and clustered cTBs, as well as cTB-clusters to distinguish pregnant women with PAS from those with placenta previa and normal placentation. ROC curves summarized in FIGS. 12A-12C demonstrated that all of single and clustered cTBs, as well as cTB-clusters met this selection criterion. A stepwise multivariate logistic regression model was then conducted (Table 3) to combine the single and clustered cTBs, as well as cTB clusters for differentiating PAS from non-PAS (placenta previa plus normal placentation) followed by the leave-one-out cross-validation, and applied this logistic regression model to an independent test set (FIG. 3).

TABLE 3
Detailed information for the multivariate logistic regression analysis.
Multivariate logistic regression results (PAS versus non-PAS)

| | | | |
|---|---|---|---|
| | Odds | 95% Confidence | |
| Logistic regression model-stepwise | ratio | Intervals (CI) | p value |

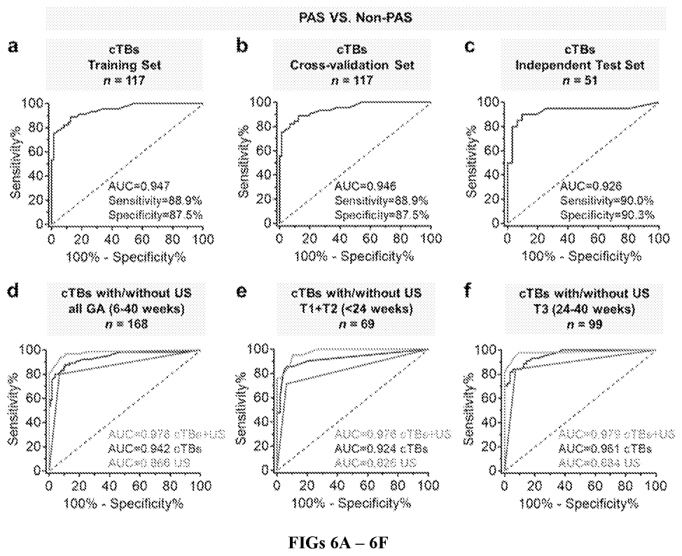

CTB assay-training cohort (FIG. 6a, AUC = 0.947)

| | | | |
|---|---|---|---|
| cTB-clusters | 1.710 | 1.264-2.314 | <0.001 |
| Single cTBs | 1.130 | 1.013-1.260 | 0.028 |

| | |
|---|---|
| Clustered cTBs | not included in the model |

CTB assay-all gestational age (FIG. 6d-blue line, AUC = 0.942)

| | | | |
|---|---|---|---|
| cTB-clusters | 1.760 | 1.356-2.284 | <0.001 |
| Single cTBs | 1.093 | 1.014-1.177 | 0.020 |

| | |
|---|---|
| Clustered cTBs | not included in the model |

cTB assay+ultrasound-all gestational age (FIG. 6d-orange line, AUC = 0.978)

| | | | |
|---|---|---|---|
| Clustered cTBs | 1.231 | 1.114-1.360 | <0.001 |
| Ultrasound | 88.8 | 18.71-421.4 | <0.001 |

| | |
|---|---|
| Single cTBs, cTB-clusters | not included in the model |

CTB assay-earlier gestational age (FIG. 6e-blue line, AUC = 0.924)

| | | | |
|---|---|---|---|
| cTB-clusters | 1.937 | 1.400-2.679 | <0.001 |

| | |
|---|---|
| Single cTBs, clustered cTBs | not included in the model |

CTB assay + ultrasound-earlier gestational age (FIG. 6e-orange line, AUC = 0.976)

| | | | |
|---|---|---|---|
| cTB-clusters | 1.888 | 1.234-2.887 | 0.003 |
| Ultrasound | 39.76 | 3.924-402.8 | 0.002 |

| | |
|---|---|
| Single cTBs, clustered cTBs | not included in the model |

CTB assay-late gestational age (FIG. 6f-blue line, AUC = 0.961)

| | | | |
|---|---|---|---|
| cTB-clusters | 2.090 | 1.311-3.331 | 0.002 |
| Single cTBs | 1.282 | 1.078-1.525 | 0.005 |

| | |
|---|---|
| Clustered cTBs | not included in the model |

cTB assay + ultrasound-late gestational age (FIG. 6f-orange line, AUC = 0.979)

| | | | |
|---|---|---|---|
| cTB-clusters | 3.085 | 1.493-6.377 | 0.002 |
| Ultrasound | 145.7 | 12.08-1758 | <0.001 |

| | |
|---|---|
| Single cTBs, clustered cTBs | not included in the model |

cTB assay + clinical factors-all gestational age (AUC = 0.978)

| Clustered cTBs | 1.231 | 1.114-1.360 | <0.001 |
|---|---|---|---|
| Ultrasound | 88.8 | 18.71-421.4 | <0.001 |
| Single cTBs, cTB-clustereds, | not included in the model | | |
| Maternal age, BMI, previous CD, | | | |
| Gravidity, Parity, IVF | | | |

For the stepwise multivariate logistic regression model, variables are entered if p<0.05, variables are removed if p>0.1. CD, cesarean delivery; BMI, body mass index; IVF, in vitro fertilization

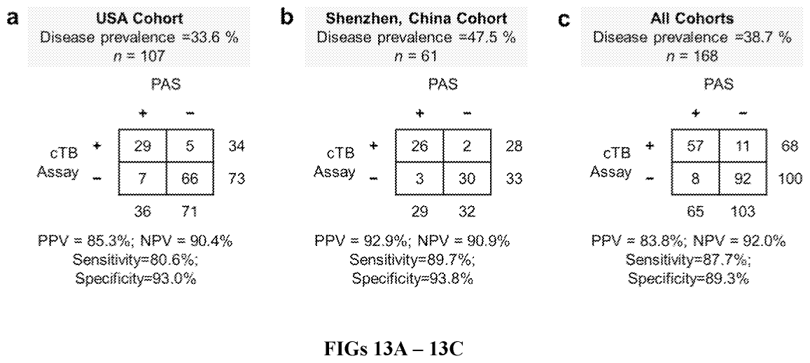

FIGS. 6A-6C summarize the diagnostic performance of the combined cTB assay for distinguishing PAS from non-PAS in the training set, after leave-one-out cross validation, and in the independent test set with areas under the curve (AUC) of 0.947 (sensitivity=88.9%, specificity=87.5%), 0.946 (sensitivity=88.9%, specificity=87.5%), and 0.926 (sensitivity=90.0%, specificity=90.3%), respectively. PAS prevalence was observed to be higher in Shenzhen cohort (47.5%) than the USA cohort (33.6%) in this study. The positive prediction value (PPV) and negative prediction value (NPV) for the cTB assay in the subpopulations of USA cohort (PPV=85.3%, NPV=90.4%) and Shenzhen cohort (PPV=92.9%, NPV=90.9%) as well as all cohorts (PPV=83.8%, NPV=92.0%) were calculated. These data are summarized in FIGS. 13A-13C.

To explore whether the cTB assay improves prediction of PAS if combined with ultrasound and other clinical risk factors listed in Table 1, such as a previous cesarean delivery (CD), maternal age, maternal body mass index (BMI), in vitro fertilization (IVF), gravidity and parity, multivariate logistic regression analysis was used to evaluate and select significant clinical predictors. The results showed that in addition to the cTB assay, ultrasound is the most statistically significant contributor associated with PAS (p<0.001, Table 3). A stepwise multivariate logistic regression model was constructed to see if the combination of the cTB assay with ultrasound improves prediction of PAS. As the comparison of ROC curves showed in FIG. 6D, the combination of cTB assay along with ultrasound achieved an AUC of 0.978, which outperformed the cTB assay alone (with AUC of 0.978 versus 0.942, p=0.052, not significant), or the ultrasound alone (with AUC of 0.978 versus 0.866, p<0.001).

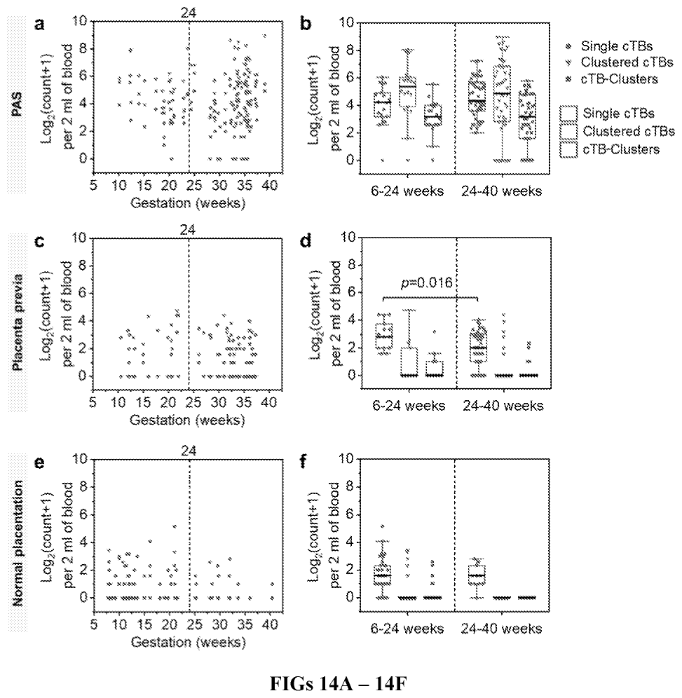

Both single and clustered cTBs can be detected throughout gestation. The counts of single and clustered cTBs, as well as cTB-clusters based on GA for each group are displayed in FIGS. 14A-14F. There was no statistically significant difference between earlier GA and late GA in the counts of single and clustered cTBs, as well as cTB-clusters in PAS and normal placentation. The statistically significant decrease was only observed for single cTBs in placenta previa. To explore whether the cTB assay performs differently in varying GA, logistic regression models (Table 3) were then conducted and comparison of ROC analysis on the subpopulations of pregnant women at earlier GA (<24 weeks) and late GA (≥24 weeks) to distinguish PAS from non-PAS, respectively. The cTB assay in the subpopulations can differentiate PAS from non-PAS regardless of GA. Again, the cTB assay can improve prediction of PAS if combined with the ultrasound in both earlier GA and late GA. In the earlier GA population (FIG. 6E), the combination of cTB assay along with ultrasound achieved an AUC of 0.976, which outperformed the cTB assay alone (with AUC of 0.976 versus 0.924, p=0.171, not significant), or the ultrasound alone (with AUC of 0.976 versus 0.826, p=0.002). In the late GA population (FIG. 6F), the combination of cTB assay along with ultrasound achieved an AUC of 0.979, which outperformed the cTB assay alone (with AUC of 0.979 versus 0.961, p=0.255, not significant), or the ultrasound alone (with AUC of 0.979 versus 0.884, p=0.001). These results show that the potential of cTB assay for the detection of PAS can be used throughout gestation, and the cTB assay is also reproducible in tight GA windows of both earlier GA and late GA.

In this study, the numbers of single (p=0.010, rₛ=0.197) and clustered cTBs (p=0.015, rₛ=0.187), as well as cTB-clusters (p=0.015, rₛ=0.187) are correlated with a previous CD. However, they are not correlated with other known risk factors for PAS including maternal age, maternal BMI, IVF, or gravidity and parity. Among all 63 cases of PAS, there are 43 (66%) patients who have both a previous CD and a previa and 5 (8%) patients have neither a previous CD nor a previa (Table 4), which are the most important two risk factors for PAS.

TABLE 4
Summary of PAS patients with/without placenta previa, previous
cesarean delivery (CD), and hysterectomy.

| Previa | Previous CD | Cases (%) | Hysterectomy | Cases |
|---|---|---|---|---|
| Yes | Yes | 43 (66%) | Yes | 32 (49%) |
| Yes | No | 10 (15%) | No | 33 (51%) |
| No | Yes | 7 (11%) | | |
| No | No | 5 (8%) | | |

| | |
|---|---|
| Total PAS | 65 |

FIGS. 6A-6F are data graphs showing receiver operating characteristic (ROC) curves of cTB assay with/without ultrasound according to an embodiment of the invention. ROC curves of cTB assay analyzed in the training set (FIG. 6A), after leave-one-out cross validation (FIG. 6B), and independent test set for distinguishing PAS from non-PAS during all GA (6-40 weeks) (FIG. 6C). Area under the curves (AUC) with the sensitivity and specificity of the assays at the optimal cutoffs are listed for each graph. Comparison of ROC curves of cTB assay with or without ultrasound for distinguishing PAS from non-PAS in all GA (6-40 weeks) (FIG. 6D), earlier GA (The first and second trimester, <24 weeks) (FIG. 6E), and late GA (≥24 weeks) (FIG. 6F). AUC for each diagnostic model was listed for each ROC curve.

FIGS. 12A-12C show receiver operating characteristic (ROC) curves of single cTBs, clustered cTBs, and cTB-clusters analyzed in different groups according to an embodiment of the invention. Counts of: single cTBs (FIG. 12A), clustered cTBs (FIG. 12B), and cTB-clusters (FIG. 12C) were analyzed in PAS versus non-PAS in all gestational ages (GA=6-40 weeks). Area under the curves (AUC) with the optimal cTB count cutoff detecting PAS, as well as the sensitivity and specificity of the assays at the optimal cutoffs are listed for each graph.

FIGS. 13A-13C are data plots showing the positive predictive values (PPV) and negative predictive values (NPV) as well as sensitivity and specificity for USA cohort, Shenzhen cohort and all cohorts according to an embodiment of the invention. The data was entered in a 2×2 table for each analysis.

FIGS. 14A-14F are data graphs showing the counts of single cTBs, clustered cTBs, and cTB-clusters based on gestational age for pregnant women according to an embodiment of the invention. FIGS. 14A and 14B show results with PAS, FIGS. 14C and 14D show results with placenta previa, and FIGS. 14E and 14F show results with normal placentation. Counts were log 2-transformed. Box plots show whiskers ranging from minima to maxima, median and 25-75% IQR. Data are expressed as Mean±SE for FIG. 14B PAS<24 weeks: single cTBs (4.0±0.3), clustered cTBs (5.1±0.5), cTB clusters (3.1±0.3); PAS≥24 weeks: single cTBs (4.5±0.2), clustered cTBs (4.6±0.4), cTB clusters (3.0±0.3); FIG. 14D placenta previa <24 weeks: single cTBs (2.9±0.6), clustered cTBs (0.8±0.4), cTB clusters (0.5±0.2); placenta previa ≥24 weeks: single cTBs (1.9±0.2), clustered cTBs (0.6±0.2), cTB clusters (0.3±0.1); and FIG. 14F normal placentation <24 weeks: single cTBs (1.7±0.2), clustered cTBs (0.5±0.2), cTB clusters (0.3±0.1); normal placentation ≥24 weeks: single cTBs (1.5±0.2), clustered cTBs (0.0±0.0), cTB clusters (0.0±0.0).

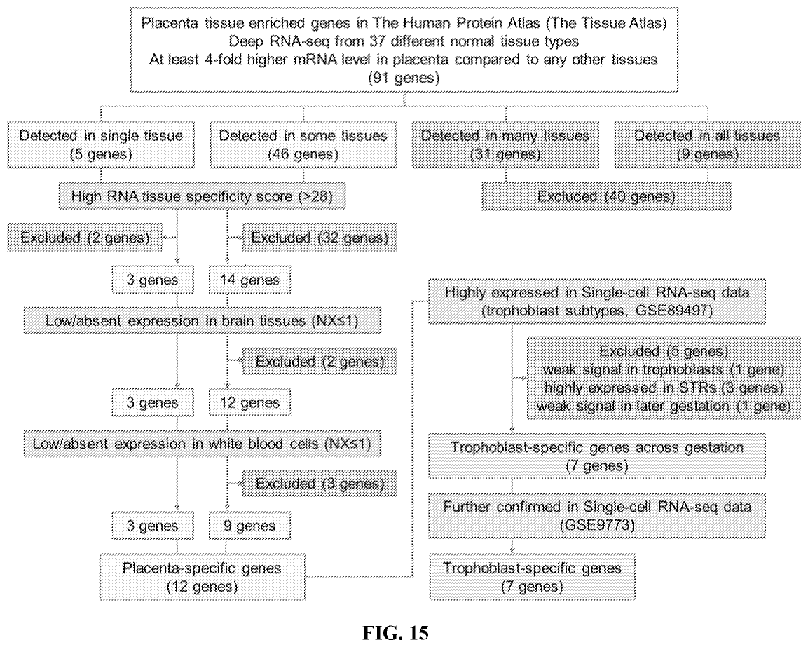

Confirming the trophoblast origin of the cTBs by detecting trophoblast-specific genes. The trophoblast origin of the cTBs was further confirmed by detecting trophoblast-specific genes. To identify trophoblast-specific genes throughout gestation, publicly available human placenta transcriptome datasets through the Human Protein Atlas was used (https://www.proteinatlas.org/humanproteome/tissue/placenia)³⁵⁻³⁷. As summarized in FIG. 15, 91 placenta enriched genes that had the highest gene expression compared to other tissues were initially identified (≥4-fold higher mRNA expression). Subsequently, to increase specificity, genes that were detected either only in placenta (5 genes) or in less than one-third of other tissue types (46 genes) were selected. A high tissue specificity (TS) score (TS>28), which is determined by fold-change between the expression in placenta to tissue with the second-highest expression level, was then used to further increase placental specificity of the placenta specific genes. Since brain and blood cells have unique datasets in the Human Protein Atlas (The Brain Atlas and The Blood Atlas, respectively), only genes that had low/absent expression in brain or WBCs (NX≤1) were selected. These selection steps resulted in a panel of 12 genes that are highly specific for the placenta (FIG. 15). To identify genes specific to the trophoblast population of the placenta, which is the population in the maternal circulation^{20,21}, a publicly available single-cell RNA sequencing data of the placenta (i.e., GSE89497³⁸) was used. Of the identified 12 placenta-specific genes, highly expressed genes in trophoblasts were selected and those also highly expressed in villous stromal cells were excluded. Trophoblast candidate genes that were highly expressed throughout gestation were selected to broaden utilization. To minimize differences due to fetal sex, trophoblast candidate genes were not sexually dimorphic³⁹. The resulting 7 trophoblast-specific genes were further confirmed in another publicly available single-cell RNA sequencing dataset of trophoblasts, GSE9773⁴⁰ (FIG. 15). The final 7 trophoblast-specific genes include chorionic somatomammotropin hormone (CSH)1, CSH2, pappalysin (PAPPA)2, pregnancy-specific beta-1-glycoprotein (PSG)1, PSG2, PSG3, PSG11 (Table 5).

TABLE 5
Detailed information for the 7 trophoblast-specific genes.

| | | | | RNA tissue | RNA tissue- |
|---|---|---|---|---|---|
| | | | | specificity | specific NX |
| Gene | Gene synonym | Gene description | Chromosome | score | (placenta) |
| PSG1 | CD66f, PBG1, | Pregnancy specific beta- | 19 | 362 | 497.6 |
| | PSBG1, PSGGA | 1-glycoprotein 1 | | | |
| PSG3 | | Pregnancy specific beta- | 19 | 146 | 495.8 |
| | | 1-glycoprotein 3 | | | |
| CSH2 | CS-2, CSB, hCS-B | Chorionic | 17 | 36 | 246.2 |
| | | somatomammotropin | | | |
| | | hormone 2 | | | |
| PAPPA2 | PAPP-A2, PAPPE, | Pappalysin 2 | 1 | 35 | 406.4 |
| | PLAC3 | | | | |
| CSH1 | CSA, CSMT, | Chorionic | 17 | 29 | 450.4 |
| | FLJ75407, hCS-A, | somatomammotropin | | | |
| | PL | hormone 1 | | | |
| PSG11 | MGC22484, | Pregnancy specific beta- | 19 | 220 | 151.1 |
| | PSG13, PSG14 | 1-glycoprotein 11 | | | |
| PSG2 | CEA, PSBG2, | Pregnancy specific beta- | 19 | 102 | 75.3 |
| | PSG1, PSGGB | 1-glycoprotein 2 | | | |

Tissue specificity score (TS) defines the fold-change between the expression level in the placenta and the tissue with the second-highest expression level. The mRNA transcript level of each gene in the placenta are shown as normalized expression (NX) values (transcript detectable level describes as NX≥1). Data are summarized based on the publicly available human placenta transcriptome datasets through the Human Protein Atlas (https://www.proteinatlas.org/humanproteome/tissue/placenia).

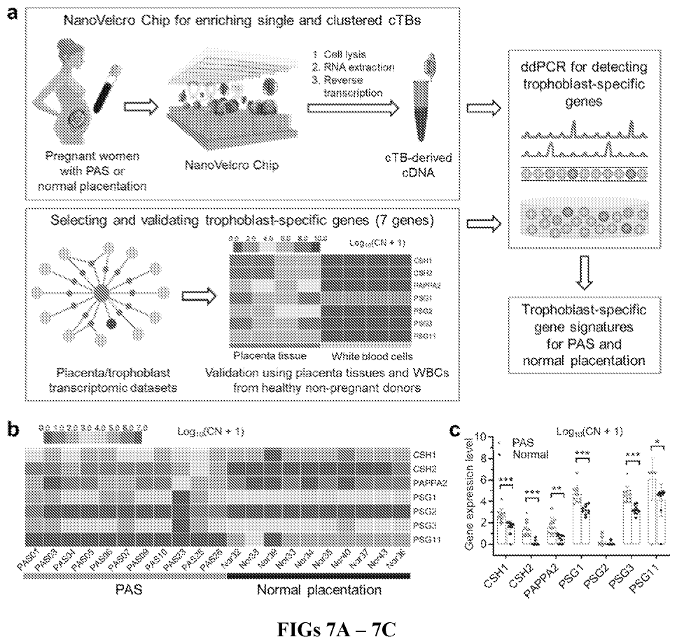

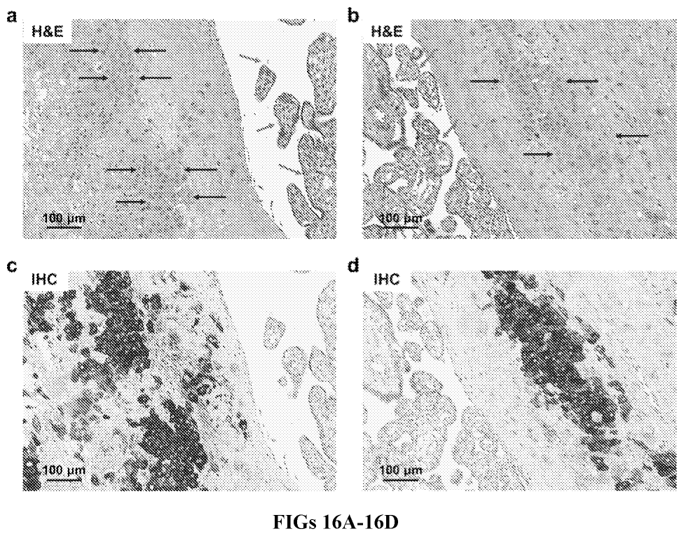

The trophoblast specificity of the 7 genes were validated using trophoblast cells from the placental tissue of PAS compared to WBCs from healthy non-pregnant female donors (FIG. 7A). Hematoxylin and Eosin (H&E) staining and HLA-G immunohistochemistry staining of placenta tissue ensured that trophoblast cells are identified and dissected for RT-ddPCR (FIGS. 16A-16D). Heat maps of placenta-derived gene signatures obtained from 4 placenta samples and 4 non-pregnant female donor WBC specimens (FIG. 7A, heat maps) demonstrates that all 7 trophoblast-specific genes are highly expressed in the trophoblast within the placenta from PAS patients and absent in WBCs. Trophoblast-specific gene expression was studied in cTBs captured on NanoVelcro Chips from 21 pregnant women including 11 with PAS and 10 with normal placentation. PBMCs were obtained from 2 mL of whole blood and processed through the NanoVelcro Chips. RNA was extracted from single and clustered cTBs captured on the NanoVelcro Chips, followed by RT-ddPCR for the trophoblast-specific gene detection (FIG. 7A). As depicted in the heat maps (FIG. 7B), signals of the 7-validated trophoblast-specific genes were identified in both pregnant women with PAS and normal placentation. The primary copy numbers were log 10-transformed for each gene. Of the 7 trophoblast-specific genes, all had significantly higher expression in PAS than those in normal placentation, except PSG2 (FIG. 7C). These results further confirmed the trophoblast origin of the single and clustered cTBs enriched by NanoVelcro Chips.

FIGS. 7A-7C are schematics and data graphs of RT-ddPCR assays for detection of trophoblast-specific genes in the cTBs captured by NanoVelcro Chips confirming trophoblast cells of placental origin according to an embodiment of the invention. FIG. 7A is a schematic illustrating the general workflow for detecting trophoblast-specific genes in cTBs captured by NanoVelcro Chips. The trophoblast-specific genes were selected from placenta transcriptome databases, validated using trophoblast cells from the placental tissue of PAS compared to white blood cells (WBCs) from healthy non-pregnant female donors. FIG. 7B shows heat maps depicting relative signal intensities for gene expression of 7 trophoblast-specific genes in the cTBs enriched from pregnant women with PAS and normal placentation. FIG. 7C is a bar graph showing differences in gene expression for 7 trophoblast-specific genes in the cTBs enriched from pregnant women with PAS and normal placentation. Primary copy numbers (CN) were log 10-transformed for each gene and False Discovery Rate (FDR) was controlled for multiple comparisons. The adjusted p value (q value) for each gene was indicated. Data are presented as means±SD. ***q<0.001, **q<0.01, *q<0.05. Clinical data for patients are listed in Table 1 and Table 2.

FIG. 15 is a flowchart for selecting placenta-specific genes and trophoblast-specific genes according to an embodiment of the invention. Placenta enriched genes (91 genes) were first identified from The Human Protein Atlas (The Tissue Atlas) database, where the transcript profiling was based on a combination of three transcriptomics datasets (HPA, GTEx, and FANTOMS), corresponding to a total of 483 samples from 37 different human normal tissue types. Placenta-specific genes were subsequently identified among the placenta enriched genes that had high expression in placenta compared to other tissues (including brain and white blood cells) (12 genes). Trophoblast-specific genes were then identified among the placenta-specific genes using single-cell RNA-sequencing datasets. A 7-gene panel that is specific to the trophoblast subpopulation across gestation was identified. Genes that have weak signal in trophoblasts, weak signal in late gestation, or high signal in villous stromal cells (STRs) were excluded. The resulting 7 genes were further confirmed and defined as trophoblast-specific genes.

FIGS. 16A-16D are representative images of Hematoxylin and Eosin (H&E) staining and immunohistochemistry (IHC) staining of placenta tissues of PAS patients according to an embodiment of the invention. FIGS. 16A and 16B are representative images of H&E staining. The arrows indicate the extravillous trophoblast cells and chronic villi. FIGS. 16C and 16D are representative images of immunohistochemistry staining of HLA-G in extravillous trophoblast cells. HLA-G negative chronic villi tissue serves as the internal negative control. Scale bar, 100 μm.

Discussion

In this study, nanostructure-embedded microchips (i.e., NanoVelcro Chips) were engineered and optimized to fulfill an unmet clinical need for the early detection of PAS. The capacity of NanoVelcro Chips to enrich and detect both single and clustered cTBs simultaneously enables accurate enumeration of single and clustered cTBs, as well as cTB-clusters. The counts of single and clustered cTBs, as well as cTB-clusters were significantly higher in women with PAS than placenta previa and normal placentation throughout gestation, and also profound in the subpopulations of both earlier GA and late GA. The enumeration of cTBs and cTB-clusters holds great promise for the early detection of PAS, and augments ultrasound screening, with implication to low resource settings. cTB-clusters, a striking characteristic that has never been described for PAS, were studied and characterized. Although cTBs have been detected in maternal blood using multiple technologies^{20,22-25}, and clustered cTBs were observed when detecting single cTBs using immunomagnetic cell sorting methods^{24,41}, the majority of current cTB detecting technologies were focused on NIPT. None of them have been developed to enrich single and clustered cTBs simultaneously for detecting PAS.

Current screening paradigms for PAS include clinical history risk stratification combined with 2D ultrasonography⁴², with MRI as an adjunct to ultrasound in cases of more severe placental invasion or cases with posterior placentation⁴. However, the imaging-based antenatal diagnosis of PAS disorders remains subjective, with accuracy dependent on the operator, similar to limitations seen for fetal anomaly screening⁷. Moreover, it was reported that MRI is often misleading when used as an adjunct to ultrasound in the management of PAS⁶, and MRI is expensive and requires expertise that is rarely available in lower resource settings. Recent population studies have shown that half to two-thirds of cases of PAS disorders remain undiagnosed before delivery^{43,44}, highlighting the crucial need to develop new technologies for prenatal detection. The instant study demonstrates a promising noninvasive technology for detecting PAS that does not rely on imaging instruments or expertise by taking advantage of the in vitro diagnostic value of NanoVelcro Chips, capable of accurately enumerating single and clustered cTBs, as well as cTB-clusters.

The combination of single and clustered cTBs, as well as cTB-clusters can differentiate PAS from placenta previa and normal placentation during pregnancy with excellent diagnostic performance throughout gestation, but most importantly, the cTB assay can also be applied early in gestation. cTBs have been identified to decline with increasing GA in normal placentation^{24,41}. In this study, no significant difference was observed between the earlier and late GA in counts of single, and clustered cTBs, as well as cTB-clusters in PAS and non-PAS groups except the single cTBs in the placenta previa group, which may be explained by the high rate of resolution of previas later in gestation. The cTB assay to distinguish PAS from non-PAS performed well throughout gestation and it also performed reproducibly in both earlier and late GA. The cTB assay provides the opportunity for the noninvasive detection of PAS earlier in gestation, with significant potential to expedite early intervention, including referral of these pregnancies to tertiary care centers and Centers of Excellence for PAS, which provides the opportunity to improve clinical outcomes⁴⁵.

In this study, the use of single and clustered cTBs, as well as cTB-clusters enriched by NanoVelcro Chips for detecting PAS was evaluated. Significantly increased numbers of single and clustered cTBs as well as cTB-clusters were observed in PAS in contrast to non-PAS. The uniqueness of the described NanoVelcro Chips stems from the use of nanostructured substrates, which allow for enhanced local topographic interactions between the nanostructured substrates and nanoscale cellular surface components (e.g., microvilli), resulting in vastly improved cell-capture affinities^{30,31}. This unique mechanism of NanoVelcro Chips makes them well-suited for capturing single cTBs and even more so for capturing cTB-clusters. In addition, the instantly disclosed NanoVelcro Chips introduce negligible perturbations to the cTB-clusters during the capture process. The prevalence of cTB-clusters with more than two cTBs attached to each other makes them more visible and less biased for enumeration. Given the streamlined workflow developed for capturing cTBs and cTB-clusters in NanoVelcro Chips, this cost-effective assay can be scaled-up in a large-scale multicenter validation study.

cTBs have been identified in maternal circulation as far back as 1893 when multinucleate fetal trophoblasts were reported at postmortem examinations of lungs from women dying as a result of eclampsia⁴⁶. Unlike the cTBs found in the pulmonary vasculature postmortem, the cTB-clusters are in the venous system, which requires passage through capillaries. Thus it remains to be determined if this clustering is unique to the type of trophoblast that has extra invasive properties leading to PAS and travels as cTB-clusters²⁴, similar to tumor cells that are more aggressive and have the capability to enter the venous system as large CTC-clusters⁴⁷. In previous studies^{25,48}, cTBs have been demonstrated to be of fetus origin as they carry both paternal and maternal alleles. In this study, it was confirmed that they are of trophoblast origin, similar to recent studies that identified cTBs to be EVTs of placental origin^{20,21}. Moreover, HLA-G expression⁴⁹ in-situ on both single and clustered cTBs supports they are all of the same immune-phenotype and derived from an EVT cell type.

This is the first description of the prevalence of cTB-clusters in pregnant women with PAS. To enable accurate cTB and cTB-cluster enumeration for detecting PAS, a nanostructure-embedded microchip that can isolate both single and clustered cTBs efficiently was engineered and optimized. Enumeration of single and clustered cTBs, as well as cTB-clusters isolated by the NanoVelcro Chips can be used for noninvasive detection of PAS throughout gestation. Also, it can be used earlier in gestation, which holds great promise to augment current diagnostic paradigms for detecting PAS. The combination of cTBs and cTB-clusters captured on the NanoVelcro Chips for detecting PAS early in gestation will enable a promising quantitative assay to serve as a noninvasive test and also as a complement to ultrasonography to improve diagnostic accuracy for PAS early in gestation.

Methods

Study design. This observational cohort study protocol was approved by the Institutional Review Boards (IRB) of University of California, Los Angeles (UCLA) (UCLA IRB #13-001264), University of Utah Health (MTA-2018-1091), Cedars-Sinai Medical Center (CSMC) (CSMC IRB #Pro00006806 and Pro00008600) and Shenzhen People's Hospital (LL-KY-2019608). All samples from pregnant women and healthy non-pregnant female donors were obtained according to protocols approved by the IRB. Written informed consent was obtained from all the participating subjects. Pregnant women aged from 18 to 45 years old with singleton intrauterine pregnancies, and GA between 6 and 40 weeks were eligible for inclusion. Women were excluded if they are diagnosed with known or suspected aneuploidy, fetal genetic/congenital anomalies or blood draw was not possible. Samples were collected between December 2017 and January 2021 during prenatal care visits. Pregnant women were classified as normal placentation, placenta previa (without placenta accreta), and PAS. PAS or placenta previa was defined and diagnosed according to current American College of Obstetricians and Gynecologists (ACOG) and Society for Maternal-Fetal Medicine (SMFM) guidelines⁴ as well as FIGO consensus guidelines⁷. The primary diagnostic modality for antenatal diagnosis was a maternal-fetal medicine obstetrics ultrasound. PAS was confirmed by histopathological diagnosis after delivery after reviewed by pathologists with expertise in gynecological and perinatal pathology as part of routine clinical care for those patients who underwent a hysterectomy. The patients who did not have a hysterectomy were confirmed PAS during the cesarean delivery⁷. An intraoperative or clinical diagnosis of PAS is made in accordance with the International FIGO classification of PAS based on general classification and grading³ (Table 4). Normal placentation had no clinical evidence of pregnancy complications or any fetal abnormalities. The pilot study was conducted at UCLA. NanoVelcro Chips were optimized using clustered cTB blood sample models and then used to isolate and detect cTBs and cTB-clusters from clinical samples for detection of PAS at both UCLA (samples collected in USA) and Shenzhen (samples collected in China). All blood samples collected at CSMC or University of Utah Health were sent to UCLA for NanoVelcro Chip assay on the day of blood draw. The sample size was calculated according to the AUC comparison between the assay and the clinical ultrasound using the paired DeLong's test. A sample size of 128 (51 cases of PAS and 77 cases of control, the ratio of sample sizes in negative/positive groups is 3/2) is expected to have 80% power to detect the difference between the AUCs for cTB assay versus ultrasound, assuming AUC=0.920 for the cTB assay, AUC=0.800 for ultrasound, when a correlation between the assays of 0.5 was assumed. The power was obtained for a two-sided test at 0.05 significance level. All laboratory samples were assayed by investigators blinded to the clinical status of the subjects.

Preparation of single and clustered JEG-3 cells. The human choriocarcinoma cell line JEG-3 was acquired from the American Type Culture Collection (ATCC, USA) and cultured in DMEM supplemented with 10% fetal calf serum in a humidified atmosphere of 5% CO₂ at 37° C. The cell line was tested and found negative for Mycoplasma contamination. A floating mixture of single and clustered JEG-3 cells were generated under a sphere-forming condition³⁴ and used to test the performance of NanoVelcro Chips for capturing both single and clustered JEG-3 cells. Briefly, logarithmic phase JEG-3 cells were digested in 0.25% trypsin, and once cells detached, digestion was terminated with serum-free culture media (SFM) composed of DMEM/F12 (1:1) basal medium (Hyclone, USA) supplemented with 20 ng/mL human epidermal growth factor (EGF) (PeproTech, USA), 20 ng/mL basic fibroblast growth factor (bFGF) (PeproTech, USA), 0.4% bovine serum albumin (BSA) (Sigma, USA), 4 μg/mL insulin, 1:50 B27 supplement (Gibco/Invitrogen, Australia) and 100 U/mL penicillin. A single-cell suspension of 5000-20000 JEG-3 cells was seeded in each well of a low adhesion 6 well plate. The medium was replaced every other day. Cell sphere-formation was observed by inverted microscopy; when cell spheres expanded 50-fold, the supernatant was collected, centrifuged at 1,000 rpm for 5 min, and the cell spheres were washed with phosphate buffer solution (PBS) twice. The live single and clustered JEG-3 cells were characterized by acquiring an optical microscope image before dye labeling. Then, the mixture of single and clustered JEG-3 cells was pre-stained with Vybrant™ DiO green fluorescent dye, and the PBMCs isolated from healthy non-pregnant female donor's whole blood were pre-stained with Vybrant™ DiD red fluorescent dye (Invitrogen, USA). The cell pre-staining process was performed in a serum-free culture medium at 37° C. for 1 h for cell-labeling before the spiking study. Excess cell-labeling dye was removed by centrifuging the labeled suspension at 1,000 rpm for 5 min and washed with PBS twice. After dye labeling, the cell mixture was resuspended with PBS. A 2.5-4 mixture of single and clustered JEG-3 cells was deposited on an ultralow-attachment culture dish⁵⁰. The population of single and clustered JEG-3 cells was characterized by acquiring fluorescence microscope images before spiking. After that, the mixture of single and clustered JEG-3 cells was re-pipetted and spiked into PBMCs to prepare a clustered cTB blood sample model for the immobilization onto a NanoVelcro Chip. The culture dish was reimaged to account for the mixture of single and clustered JEG-3 cells that remained attached to the surface. By post-processing these microscope images, single JEG-3 cells and clustered JEG-3 cells within each cluster that were ultimately spiked into female donor's whole blood could be accurately enumerated.

NanoVelcro Chip optimization. The clustered cTB blood sample models containing both single and clustered JEG-3 cells were incubated with biotin-conjugated anti-EpCAM for 30 min at room temperature, and excess antibody was removed by PBS washing and centrifuging the suspension at 1000 rpm for 5 min. The samples were re-suspended in PBS (200 μL) and injected to the NanoVelcro Chips at flow rates of 0.1, 0.2, 0.5, 1.0, and 2.0 mL/h, respectively. The single and clustered JEG-3 cells captured in NanoVelcro Chips were fixed with 4.0% formaldehyde (PFA, Electron Microscopy Sciences) in PBS (200 μL). After nuclear staining with DAPI, all the stained chips were scanned and imaged under a fluorescence microscope (Nikon 90i). The single and clustered JEG-3 cells captured on the chips were then counted.

SEM sample preparation and imaging. A mixture of single and clustered JEG-3 cells was captured onto the NanoVelcro Chips. After separating the PDMS tops from the chips, the single and clustered JEG-3 cells were first fixed in 4% glutaraldehyde and then in 1% osmium tetroxide (both diluted in 0.1 M sodium cacodylate) for 1 hour in each. After fixation, cells were dehydrated in 50%, 70%, 80%, 95%, and 100% ethanol (200 proof) solutions successively for 15 min in each. The samples were dried overnight and then sputter-coated with gold at room temperature. Prepared samples were imaged using a Zeiss Supra 55VP field emission scanning electron microscope at UCLA.

Clinical sample collection and blood processing. Blood samples were collected in the Department of Obstetrics and Gynecology, David Geffen School of Medicine, UCLA; Los Angeles and the Center for Fetal Medicine and Women's Ultrasound; Los Angeles; Department of Obstetrics and Gynecology, Cedars-Sinai Medical Center, Los Angeles; Department of Obstetrics & Gynecology, University of Utah Health, Salt Lake City, UT, USA; Department of Obstetrics and Gynecology, Shenzhen People's Hospital, China. Clinical information was abstracted from all subject charts. Peripheral blood samples were collected in 10 mL acid citrate dextrose (ACD) vacutainer tubes and were processed on the same day. The PBMCs were isolated in 15 min by using SepMate™ PBMC Isolation Tubes (STEMCELL Technologies, USA) and aliquoted following a previously published protocol⁵¹. PBMCs from 2 mL of whole blood were run through the NanoVelcro Chips using the optimized protocol. The streamlined workflow is composed of the following steps: (i) capturing cTB cells/clusters onto NanoVelcro Chips (30 min), (ii) overnight standard immunostaining, and (iii) imaging/counting by a fluorescent microscope (30 min).

Immunostaining and identification of single and clustered cTBs. The samples were run through NanoVelcro Chips under optimized conditions. After 4% PFA fixation, the immobilized cells were subjected to 4-color ICC staining with DAPI (#422801, BioLegend, USA), anti-cytokeratin 7 antibody (Rabbit polyclonal IgG) (#ab53123, abcam, USA), anti-HLA-G antibody (4H84) (#ab52455, abcam, USA), and anti-CD45 monoclonal antibody (YAML501.4) (#MA5-17687, Thermofisher, USA) for identification of cTBs (DAPF/CK7+/HLA-G+/CD45−) and cTB-clusters. Immobilized cells were imaged using the Nikon Ni fluorescence microscope with NIS-Element imaging software (Nikon Eclipse Ti2). An automatic scan was carried out under 20× magnification with DAPI, FITC, TRITC, and Cy5 channels corresponding to nuclear, CK7, HLA-G, and CD45 staining, respectively. Micrographic features of candidate cTBs were reviewed to ensure consistency with epithelial as opposed to the hematologic origin. When analyzing the multi-channel ICC micrographs, WBCs were defined as round/ovoid cells (DAPI+/CK7−/HLA-G−/CD45+); and cTBs were defined as round/ovoid cells (DAPI+/CK7+/HLA-C1+/CD45−).

Pathological examination of placenta tissues. Pathological examination including Hematoxylin and Eosin (H&E) and immunohistochemistry (IHC) staining of the placenta tissues obtained from PAS patients was performed independently by pathologists with expertise in gynecological and perinatal pathology at UCLA. All of the placenta tissues were fixed in 10% neutral formalin for 24-48 h and embedded in paraffin according to the standard operating procedure (SOP) for tissue in the pathology department at UCLA. Serial 4 μm-thick tissue sections from formalin-fixed paraffin-embedded (FFPE) blocks were cut and mounted on poly-L-lysine coated glass slides. Standard IHC staining on 4-μm-thick tissue sections was performed on Ventana Benchmark ULTRA Slide Stainer according to a protocol optimized for the HLA-G antibody. The IHC analyses for HLA-G was performed to differentiate between villous trophoblasts (cytotrophoblasts and syncytiotrophoblasts) and EVTs. Positive HLA-G staining confirmed the identity of EVTs.

Trophoblast-specific gene validation on placenta tissues. Trophoblast-specific gene expression in placenta tissue was performed to validate the selected trophoblast-specific gene panel. The unstained FFPE placenta tissue slides were deparaffinized and macrodissected to enrich the trophoblasts in the placenta tissues. The target trophoblastic areas in each slide were identified and marked under microscopy in the corresponding H&E and IHC stained slides from the same FFPE block by pathologists. Total RNA was extracted from the enriched placenta tissues using Qiagen (Dusseldorf, Germany) RNeasy FFPE kit. Then the complementary DNA (cDNA) was synthesized using a Thermo Scientific Maxima H Minus Reverse Transcriptase Kit according to the manufacturer's protocols. cDNA was then tested for trophoblast-specific gene transcripts using duplex ddPCR in each tube with two fluorescence filters (i.e. FAM and VIC). Predesigned Taqman assays (Thermofisher Scientific) containing primers and probes for each gene were used in the ddPCR following manufacturer's protocols. ddPCR experiments were performed on a QX200 system (Bio-Rad Laboratories, Inc.) according to the manufacturer's protocols. Data were analyzed using the QuantaSoft™ software to quantify the corresponding copy numbers of gene transcripts detected in each assay.

Trophoblast-specific gene detection on the isolated single and clustered cTBs. NanoVelcro Chips were used for isolating the single and clustered cTBs from pregnant women. To extract RNA from the single and clustered cTBs captured on NanoVelcro Chips, on-chip lysis of cells was performed by introducing 600 μL of TRIzol solution (Zymo Research, USA) and 600 μL of anhydrous ethanol (Sigma-Aldrich) sequentially through the NanoVelcro Chips. The effluent solution was collected in a 2.0 mL RNase-free Eppendorf tube at the same time. Then, RNA was purified using a Direct-zol™ RNA MicroPrep Kit (Zymo Research). cDNA was synthesized using a Thermo Scientific Maxima H Minus Reverse Transcriptase Kit according to the manufacturer's instructions. cDNA was then tested for the trophoblast-specific gene using duplex ddPCR in each tube with two fluorescence filters (i.e. FAM and VIC). RT-ddPCR experiments were performed using the same protocol as that used for the placenta tissues.

Statistical analysis. Multiple comparisons of cTB enumeration data among different groups were evaluated using one-way ANOVA after log 2-transformation. Comparisons of the enumeration data between earlier GA and late GA were evaluated using Mann-Whitney test. Chi-square test was used for comparison of the detection rates of samples for cTBs and cTB-clusters in each group. Comparisons in Table 1 were evaluated using Mann-Whitney test or the Chi-square test. The Spearman's rank-order correlation was used to analyze the correlation of the cTB enumeration and demographic information. Multiple T-tests were used to compare the differences of trophoblast-gene expression in different groups. Benjamani, Krieger, and Yekutieli FDR were used for multiple testing correction across the set of genes with the maximum desired FDR of 1%. Logistic regression model, comparison of ROC curves, and single ROC curves were conducted using MedCalc software. The optimal cutpoints were calculated to maximize sensitivity and specificity. Leave-one-out cross validation for the logistic regression model was conducted in R studio. All the other statistical tests in this study were performed using the Graphpad Prism 9 (https://www.graphpad.com/). All tests are two-sided and p-value or q-value<0.05 is considered significant.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. Moreover, features described in connection with one embodiment of the invention may be used in conjunction with other embodiments, even if not explicitly stated above. It is therefore to be understood that, within the scope of the claims and their equivalents, the invention may be practiced otherwise than as specifically described.

### REFERENCES

1. Einerson, B. D., Comstock, J., Silver, R. M., Branch, D. W., Woodward, P. J. & Kennedy, A. Placenta accreta spectrum disorder: Uterine dehiscence, not placental invasion. Obstet. Gynecol. 135, 1104-1111 (2020).
2. Silver, R. M. & Branch, D. W. Placenta accreta spectrum. N. Engl. J. Med. 378, 1529-1536 (2018).
3. Jauniaux, E. et al. FIGO classification for the clinical diagnosis of placenta accreta spectrum disorders. Int. J. Gynaecol. Obstet. 146, 20-24 (2019).
4. Society of Gynecologic, O. et al. Placenta accreta spectrum. Am. J. Obstet. Gynecol. 219, B2-B16 (2018).
5. Berkley, E. M. & Abuhamad, A. Z. Prenatal diagnosis of placenta accreta: Is sonography all we need? J. Ultrasound Med. 32, 1345-1350 (2013).
6. Einerson, B. D., Rodriguez, C. E., Kennedy, A. M., Woodward, P. J., Donnelly, M. A. & Silver, R. M. Magnetic resonance imaging is often misleading when used as an adjunct to ultrasound in the management of placenta accreta spectrum disorders. Am. J. Obstet. Gynecol. 218, 618.e611-618.e617 (2018).
7. Jauniaux, E. et al. FIGO consensus guidelines on placenta accreta spectrum disorders: Prenatal diagnosis and screening. Int. J. Gynaecol. Obstet. 140, 274-280 (2018).
8. Jauniaux, E. & Silver, R. M. Moving from intra partum to prenatal diagnosis of placenta accreta: A quarter of a century in the making but still a long road to go. BJOG: Int. J. Obstet. Gy. 124, 96 (2017).
9. Bowman, Z. S. et al. Accuracy of ultrasound for the prediction of placenta accreta. Am. J. Obstet. Gynecol. 211, 177.e171-177.e177 (2014).
10. Philips, J. & Abuhamad, A. Diagnosing placenta accreta spectrum with prenatal ultrasound. OBG Manag. 30, 34-36, 38-44 (2018).
11. Shainker, S. A. et al. Placenta accreta spectrum: Biomarker discovery using plasma proteomics. Am. J. Obstet. Gynecol. 223, 433 e431-433 e414 (2020).
12. Bartels, H. C., Postle, J. D., Downey, P. & Brennan, D. J. Placenta accreta spectrum: A review of pathology, molecular biology, and biomarkers. Dis. Markers 2018, U.S. Pat. No. 1,507,674 (2018).
13. American College of, O. et al. Levels of maternal care. Am. J. Obstet. Gynecol. 212, 259-271 (2015).
14. Clapp, M. A., James, K. E. & Kaimal, A. J. The effect of hospital acuity on severe maternal morbidity in high-risk patients. Am. J. Obstet. Gynecol., 10.1016/j.ajog.2018.04.015, 111.e111-111.e117 (2018).
15. Shamshirsaz, A. A. et al. Multidisciplinary team learning in the management of the morbidly adherent placenta: Outcome improvements over time. Am. J. Obstet. Gynecol. 216, 612.e611-612.e615 (2017).
16. Shamshirsaz, A. A. et al. Maternal morbidity in patients with morbidly adherent placenta treated with and without a standardized multidisciplinary approach. Am. J. Obstet. Gynecol. 212, 218.e211-218.e219 (2015).
17. Jauniaux, E., Collins, S. & Burton, G. J. Placenta accreta spectrum: Pathophysiology and evidence-based anatomy for prenatal ultrasound imaging. Am. J. Obstet. Gynecol. 218, 75-87 (2018).
18. Wu, S., Kocherginsky, M. & Hibbard, J. U. Abnormal placentation: Twenty-year analysis. Am. J. Obstet. Gynecol. 192, 1458-1461 (2005).
19. Munoz, L. A., Mendoza, G. J., Gomez, M., Reyes, L. E. & Arevalo, J. J. Anesthetic management of placenta accreta in a low-resource setting: A case series. Int. J. Obstet. Anesth. 24, 329-334 (2015).
20. Breman, A. M. et al. Evidence for feasibility of fetal trophoblastic cell-based noninvasive prenatal testing. Prenat. Diagn. 36, 1009-1019 (2016).
21. Hatt, L. et al. Characterization of fetal cells from the maternal circulation by microarray gene expression analysis—could the extravillous trophoblasts be a target for future cell-based non-invasive prenatal diagnosis? Fetal Diagn. Ther. 35, 218-227 (2014).
22. Guetta, E., Gutstein-Abo, L. & Barkai, G. Trophoblasts isolated from the maternal circulation: In vitro expansion and potential application in non-invasive prenatal diagnosis. J. Histochem. Cytochem. 53, 337-339 (2005).
23. Kolvraa, S. et al. Genome-wide copy number analysis on DNA from fetal cells isolated from the blood of pregnant women. Prenat. Diagn. 36, 1127-1134 (2016).
24. Vossaert, L. et al. Validation studies for single circulating trophoblast genetic testing as a form of noninvasive prenatal diagnosis. Am. J. Hum. Genet. 105, 1262-1273 (2019).
25. Hou, S. et al. Imprinted NanoVelcro microchips for isolation and characterization of circulating fetal trophoblasts: Toward noninvasive prenatal diagnostics. ACS Nano 11, 8167-8177 (2017).
26. Moser, G., Weiss, G., Gauster, M., Sundl, M. & Huppertz, B. Evidence from the very beginning: Endoglandular trophoblasts penetrate and replace uterine glands in situ and in vitro. Hum. Reprod. 30, 2747-2757 (2015).
27. Wang, S. et al. Three-dimensional nanostructured substrates toward efficient capture of circulating tumor cells. Angew. Chem. Int. Ed. 48, 8970-8973 (2009).
28. Wang, S. et al. Highly efficient capture of circulating tumor cells by using nanostructured silicon substrates with integrated chaotic micromixers. Angew. Chem. Int. Ed. 50, 3084-3088 (2011).
29. Chen, J. F. et al. Clinical applications of NanoVelcro rare-cell assays for detection and characterization of circulating tumor cells. Theranostics 6, 1425-1439 (2016).
30. Dong, J. et al. Nanostructured substrates for detection and characterization of circulating rare cells: From materials research to clinical applications. Adv. Mater. 32, 1903663 (2020).
31. Jan, Y. J. et al. NanoVelcro rare-cell assays for detection and characterization of circulating tumor cells. Adv. Drug Deliv. Rev. 125, 78-93 (2018).
32. Goldman-Wohl, D. S., Ariel, I., Greenfield, C., Hanoch, J. & Yagel, S. HLA-G expression in extravillous trophoblasts is an intrinsic property of cell differentiation: A lesson learned from ectopic pregnancies. Mol. Hum. Reprod. 6, 535-540 (2000).
33. Craenmehr, M. H. C. et al. Increased HLA-G expression in term placenta of women with a history of recurrent miscarriage despite their genetic predisposition to decreased HLA-G levels. Int. J. Mol. Sci. 20, 625 (2019).
34. Cai, J. et al. Isolation, culture and identification of choriocarcinoma stem-like cells from the human choriocarcinoma cell-line JEG-3. Cell. Physiol. Biochem. 39, 1421-1432 (2016).
35. Uhlen, M. et al. Tissue-based map of the human proteome. Science 347, 1260419 (2015).
36. Yu, N. Y. et al. Complementing tissue characterization by integrating transcriptome profiling from the Human Protein Atlas and from the FANTOMS consortium. Nucleic Acids Res. 43, 6787-6798 (2015).
37. Fagerberg, L. et al. Analysis of the human tissue-specific expression by genome-wide integration of transcriptomics and antibody-based proteomics. Mol. Cell. Proteomics 13, 397-406 (2014).
38. Liu, Y. et al. Single-cell RNA-seq reveals the diversity of trophoblast subtypes and patterns of differentiation in the human placenta. Cell Res. 28, 819-832 (2018).
39. Gonzalez, T. L. et al. Sex differences in the late first trimester human placenta transcriptome. Biol. Sex Differ. 9, 4 (2018).
40. Bilban, M. et al. Identification of novel trophoblast invasion-related genes: Heme oxygenase-1 controls motility via peroxisome proliferator-activated receptor gamma. Endocrinology 150, 1000-1013 (2009).
41. Vossaert, L. et al. Reliable detection of subchromosomal deletions and duplications using cell-based noninvasive prenatal testing. Prenat. Diagn. 38, 1069-1078 (2018).
42. D'Antonio, F., lacovella, C. & Bhide, A. Prenatal identification of invasive placentation using ultrasound: Systematic review and meta-analysis. Ultrasound Obstet. Gynecol. 42, 509-517 (2013).
43. Bailit, J. L. et al. Morbidly adherent placenta treatments and outcomes. Obstet. Gynecol. 125, 683-689 (2015).
44. Thurn, L. et al. Abnormally invasive placenta-prevalence, risk factors and antenatal suspicion: Results from a large population-based pregnancy cohort study in the Nordic countries. BJOG: Int. J. Obstet. Gy. 123, 1348-1355 (2016).
45. Shamshirsaz, A. A., Fox, K. A., Erfani, H. & Belfort, M. A. The role of centers of excellence with multidisciplinary teams in the management of abnormal invasive placenta. Clin. Obstet. Gynecol. 61, 841-850 (2018).
46. Beaudet, A. L. Using fetal cells for prenatal diagnosis: History and recent progress. Am. J. Med. Genet. C Semin. Med. Genet. 172, 123-127 (2016).
47. Au, S. H. et al. Clusters of circulating tumor cells traverse capillary-sized vessels. Proc. Natl. Acad. Sci. U.S.A. 113, 4947-4952 (2016).
48. Mouawia, H. et al. Circulating trophoblastic cells provide genetic diagnosis in 63 fetuses at risk for cystic fibrosis or spinal muscular atrophy. Reprod. Biomed. Online 25, 508-520 (2012).
49. van Wijk, I. J. et al. HLA-G expression in trophoblast cells circulating in maternal peripheral blood during early pregnancy. Am. J. Obstet. Gynecol. 184, 991-997 (2001).
50. Sarioglu, A. F. et al. A microfluidic device for label-free, physical capture of circulating tumor cell clusters. Nat. Methods 12, 685-691 (2015).
51. Dong, J. et al. Covalent chemistry on nanostructured substrates enables noninvasive quantification of gene rearrangements in circulating tumor cells. Sci. Adv. 5, eaav9186 (2019).

1. A method for determining whether a subject has a placenta accrete spectrum (PAS) disorders, comprising:
isolating at least one of a plurality of single circulating trophoblasts (cTBs) and a plurality of clustered circulating trophoblasts (cTBs) from a blood sample from said subject;
detecting at least one of a plurality of isolated single cTBs and a plurality of isolated clustered cTBs from said blood sample;
determining at least one of a number of single cTBs from said plurality of isolated single cTBs and a number of clustered cTBs from said plurality of isolated clustered cTBs, wherein a presence of at least one of a predetermined amount of single cTBs and a predetermined amount of clustered cTBs is indicative of an increased probability of said subject having said PAS disorders; and
providing a probability of said subject having said PAS disorders based on said determining at least one of a number of single cTBs from said plurality of isolated single cTBs and a number of clustered cTBs from said plurality of isolated clustered cTBs.

2. The method of claim 1, wherein said predetermined amount of clustered cTBs is 5 clustered cTBs per milliliter of said blood sample, and wherein said predetermined amount of single cTBs is 2 single cTBs per milliliter of said blood sample.

3. The method of claim 1, further comprising:
isolating said plurality of single circulating trophoblasts (cTBs) and said plurality of clustered circulating trophoblasts (cTBs) from said blood sample;
detecting said plurality of isolated single cTBs and said plurality of isolated clustered cTBs from said blood sample;
determining a number of total cTBs from said plurality of isolated single cTBs and from said plurality of isolated clustered cTBs, wherein a presence of at least a predetermined amount of total cTBs is indicative of an increased probability of said subject having said PAS disorders; and
providing a probability of said subject having said PAS disorders based on said determining said number of total cTBs.

4. The method of claim 3, wherein said predetermined amount is 10 total cTBs per milliliter of said blood sample.

5.-8. (canceled)

9. The method of claim 1, further comprising assaying at least one of said plurality of isolated single cTBs and said plurality of isolated clustered cTBs for expression of a biomarker specific to an extra-villous trophoblast.

10. The method of claim 1, wherein said isolating at least one of a plurality of single circulating trophoblasts (cTBs) and a plurality of clustered circulating trophoblasts (cTBs) from said blood sample comprises at least one of capturing said plurality of single circulating trophoblasts (cTBs) and capturing said plurality of clustered circulating trophoblasts (cTBs) on a nanostructure-embedded microchip.

11.-20. (canceled)

21. A method for capturing at least one of a plurality of single circulating trophoblasts (cTBs) and a plurality of clustered circulating trophoblasts (cTBs) from a blood sample from a subject, comprising:
contacting said blood sample from said subject with a nanostructure-embedded chip such that at least one of said plurality of single circulating trophoblasts (cTBs) and said plurality of clustered circulating trophoblasts (cTBs) from said blood sample are captured by said nanostructure-embedded chip; and
detecting said plurality of captured single cTBs and said plurality of captured clustered cTBs from said blood sample.

22. The method of claim 21, further comprising determining a number of total cTBs from said plurality of captured single cTBs and from said plurality of captured clustered cTBs, wherein a presence of at least a predetermined amount of total cTBs is indicative of an increased probability of said subject having a placenta accrete spectrum (PAS) disorder.

23. The method of claim 22, wherein said predetermined amount of clustered cTBs is 5 clustered cTBs per milliliter of said blood sample, and wherein said predetermined amount of single cTBs is 2 single cTBs per milliliter of said blood sample.

24. The method of claim 22, further comprising:
capturing said plurality of single circulating trophoblasts (cTBs) and said plurality of clustered circulating trophoblasts (cTBs) from said blood sample;
detecting said plurality of captured single cTBs and said plurality of captured clustered cTBs from said blood sample;
determining a number of total cTBs from said plurality of isolated single cTBs and from said plurality of isolated clustered cTBs, wherein a presence of at least a predetermined amount of total cTBs is indicative of an increased probability of said subject having said PAS disorders; and
providing a probability of said subject having said PAS disorders based on said determining said number of total cTBs.

25.-27. (canceled)

28. The method of claim 21, further comprising assaying at least one of said plurality of isolated single cTBs and said plurality of isolated clustered cTBs for expression of a biomarker specific to an extra-villous trophoblast.

29. The method of claim 21, wherein said nanostructure-embedded chip comprises:
a substrate comprising a plurality of nanowires; and
a chaotic mixer overlayed on said substrate.

30. The method of claim 29, wherein said plurality of nanowires comprise a plurality of binding agents, said plurality of binding agents specific to an extra-villous trophoblast biomarker.

31. (canceled)

32. A kit for capturing at least one of a plurality of single circulating trophoblasts (cTBs) and a plurality of clustered circulating trophoblasts (cTBs) from a blood sample, comprising:
a nanostructure-embedded chip;
instructions for capturing at least one of said plurality of single circulating trophoblasts (cTBs) and said plurality of clustered circulating trophoblasts (cTBs) from said blood sample by contacting said blood sample with said nanostructure-embedded chip; and
a plurality of reagents for detecting said plurality of captured single cTBs and said plurality of captured clustered cTBs from said blood sample.

33. The kit of claim 32, wherein said instructions for capturing at least one of said plurality of single circulating trophoblasts (cTBs) and said plurality of clustered circulating trophoblasts (cTBs) from said blood sample comprise passing said blood sample through said nanostructure-embedded chip at a flow rate of between 0.2 ml/h to 1.0 ml/h.

34. The kit of claim 33, wherein said plurality of reagents for detecting said plurality of captured single cTBs and said plurality of captured clustered cTBs from said blood sample comprise a reagent for detecting a expression of a biomarker specific to an extra-villous trophoblast.

35. The kit of claim 32, further comprising instructions for determining a number of total cTBs from said plurality of captured single cTBs and from said plurality of captured clustered cTBs, wherein a presence of at least a predetermined amount of total cTBs is indicative of an increased probability of said subject having a placenta accrete spectrum (PAS) disorder.

36.-37. (canceled)

38. The kit of claim 32, wherein said nanostructure-embedded chip comprises:
a substrate comprising a plurality of nanowires; and
a chaotic mixer overlayed on said substrate.

39. The kit of claim 38, wherein said plurality of nanowires comprise a plurality of binding agents, said plurality of binding agents specific to an extra-villous trophoblast biomarker.
